(19) United States
(12) Patent Application Publication
(10) Pub. No.: US 2026/0347387 A1
(43) Pub. Date: Nov. 5, 2026

(54) Lipid-Gold Salt Molecules and Formulation in Lipid Nanoparticle Delivery Systems

(71) Applicant: NORTHWESTERN UNIVERSITY, Evanston, IL (US)

(72) Inventors: C. Shad Thaxton, Chicago, IL (US); Andrea E. Calvert, Chicago, IL (US); Sonbinh T. Nguyen, Evanston, IL (US); Aliakbar Mohammadlou, Chicago, IL (US); Jonathan S. Rink, Park Ridge, IL (US); Sohia Lamperis, Evanston, IL (US)

(21) Appl. No.: 19/486,837

(22) PCT Filed: May 31, 2024
(86) PCT No.: PCT/US2024/032085

§ 371 Date: Nov. 21, 2025

Related U.S. Application Data
(60) Provisional application No. 63/470,075, filed on May 31, 2023.

(51) Int. Cl. C07J 31/00; A61K 9/51; A61K 31/7135; A61K 33/242; A61P 35/00; C07F 9/50

(52) U.S. Cl. CPC C07J 31/003; A61K 9/5123; A61K 9/5169; A61K 9/5192; A61K 31/7135; A61K 33/242; A61P 35/00; C07F 9/5045; C07F 9/5054

(57) ABSTRACT

Disclosed herein are lipid-gold salt molecules, nanoparticles and microparticles containing these molecules, and methods of making and using the same.

### STATEMENT OF GOVERNMENT INTEREST

This invention was made with government support under grant number 1U54AR079795-01 awarded by the Department of Health and Human Services, National Institutes of Health, National Institute of Arthritis and Musculoskeletal and Skin Diseases. The government has certain rights in the invention.

### BACKGROUND

Gold salts, such as auranofin, are FDA-approved therapies for inflammatory diseases. However, large doses of these drugs may be needed to see a desired effect, and many common modes of administration do not specifically target gold salts to cells or tissues of interest, which results in side effects. There is a need for gold salt molecules that can be systemically delivered in lipid nanoparticle (LN) and synthetic lipoprotein nanoparticles (SLN), such as high-density lipoprotein-like nanoparticles (HDL NP), whereupon specific cellular targeting of the drug can be achieved. For instance, scavenger receptor type B1 (SR-B1) is a cell-surface-receptor expressed by cancer and immune cells. The SR-B1 receptor binds with high affinity to anionic LNs and SLNs. As such, drugs formulated in LNs and SLNs are more potent than approved therapeutics for inflammation and as cancer therapeutics. In short, targeted delivery and more potent efficacy are powerful benefits of gold salt molecules formulated in lipid nanoparticle systems that meet the tremendous unmet needs of patients with incalcitrant and untreatable inflammatory and cancer diseases, among other indications (e.g., antiparasitic and antimicrobial agents).

### SUMMARY

The present disclosure provides compounds, nanoparticles and microparticles, compositions comprising the compounds/particles, and methods for making and using all of the foregoing. Exemplary embodiments are summarized below, and also throughout the description and claims.

Provided herein are compounds, or pharmaceutically acceptable salt thereof, having the structure of Formula I: R¹—S—Au—P(R²)₃ (I), wherein R¹ is a steroidal moiety or a lipid moiety; and each R² is independently C₁₋₆ alkyl or C₆₋₁₀ aryl.

Also provided are compounds having a structure as shown in Table A, and pharmaceutically acceptable salts thereof.

Also provided are microparticles and nanoparticles comprising a compound or salt disclosed herein (e.g., a compound of Formula I or as shown in Table A, or a pharmaceutically acceptable salt thereof) and a lipid nanoparticle (LN) or a synthetic lipoprotein nanoparticle (SLN). In various aspects, provided are microparticles and nanoparticles wherein the synthetic lipoprotein nanoparticle (SLN) is a high-density lipoprotein-like nanoparticle (HDL-NP) comprising: (a) an organic or an inorganic nanoparticle core; (b) a shell surrounding and/or attached to the core wherein the shell comprises one or more apolipoproteins and one or more phospholipids; and (c) wherein the compound or salt disclosed herein (e.g., a compound of Formula I or as shown in Table A, or a pharmaceutically acceptable salt thereof) is associated with one or more of the core or shell.

Also provided are pharmaceutical compositions comprising a compound or salt disclosed herein (e.g., a compound of Formula I or as shown in Table A, or a pharmaceutically acceptable salt thereof), and/or microparticles or nanoparticles disclosed herein, and a pharmaceutically acceptable carrier or excipient.

Further provided are methods of treating or preventing a disease or disorder comprising administering to a subject in need thereof a therapeutically effective amount of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein. In various aspects, provided are uses of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein for the treatment or prevention of a disease or disorder, and uses of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein in the manufacture of a medicament for the treatment or prevention of a disease or disorder. In various aspects, the disease or disorder is an inflammatory disease, infectious disease, or cancer. Also provided are methods of reducing inflammation comprising administering to a subject in need thereof a therapeutically effective amount of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein. Also provided are methods of inhibiting neoplastic cell growth comprising administering to a subject in need thereof a therapeutically effective amount of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein.

Also provided are processes of synthesizing a compound of Formula (I): R¹—S—Au—P(R²)₃ (I), comprising admixing a compound of Formula (I′): R¹—SH (I′) and a compound of Formula (I″): X—Au—P(R²)₃ (I″) in the presence of a base to form the compound of Formula (I), wherein R¹ is a steroidal moiety or a lipid moiety; each R² is independently C₁₋₆ alkyl or C₆₋₁₀ aryl; and X is a halogen ion.

Also provided are processes of synthesizing microparticles and nanoparticles disclosed herein, comprising: (i) admixing one or more gold nanoparticles (AuNPs) and one or more apolipoproteins to form one or more Apo-AuNPs; (ii) incubating the one or more Apo-AuNPs; (iii) admixing the one or more Apo-AuNPs and a compound of Formula (I′): R¹—SH (I′) to form one or more R¹S-Apo-AuNPs; (iv) incubating the one or more R¹S-Apo-AuNPs; and (v) admixing the one or more R¹S-Apo-AuNPs and a compound of Formula (I″): X—Au—P(R²)₃ (I″) in the presence of a base to form the microparticle or nanoparticle, wherein X is a halogen ion.

Also provided are processes of synthesizing microparticles and nanoparticles disclosed herein, comprising: (i) admixing one or more gold nanoparticles (AuNPs) and one or more apolipoproteins in a solvent to form one or more Apo-AuNPs; (ii) incubating the one or more Apo-AuNPs; (iii) admixing the one or more Apo-AuNPs and one or more phospholipids in an organic solvent to form one or more PL-Apo-AuNPs; (iv) admixing the one or more PL-Apo-AuNPs and a compound of Formula (I) in the organic solvent; and (v) removing the organic solvent to form the microparticle or nanoparticle.

Also provided are processes of synthesizing microparticles and nanoparticles disclosed herein, comprising: (i) admixing one or more gold nanoparticles (AuNPs) and one or more apolipoproteins in a solvent to form one or more Apo-AuNPs; (ii) incubating the one or more Apo-AuNPs; (iii) admixing the one or more Apo-AuNPs and one or more phospholipids to form one or more PL-Apo-AuNPs; (iv) incubating the one or more one or more PL-Apo-AuNPs; (v) optionally repeating steps (ii) and (iii) to incorporate one or more additional phospholipids into the PL-Apo-AuNPs; and (iv) admixing the one or more PL-Apo-AuNPs and aqueous KCN to form the microparticle or nanoparticle.

Also provided are processes of synthesizing microparticles and nanoparticles disclosed herein, comprising admixing a dendritic scaffold, an apolipoprotein, a sterol salt, a phospholipid, and a compound of Formula (I) to form the microparticle or nanoparticle.

Also provided are processes of synthesizing microparticles and nanoparticles disclosed herein, wherein assembly of the microparticle or nanoparticle occurs in vivo. In various aspects, the one or more apolipoproteins are native apolipoproteins. In various aspects, the native apolipoproteins are not administered to a subject. In some instances, synthesizing the microparticle or nanoparticle comprises administering at least the following components to a mammalian subject: an organic or an inorganic nanoparticle core; and at least one compound having the structure of Formula (I) or as recited in Table A; wherein assembly of the components into the microparticle or nanoparticle occurs in vivo in the mammalian subject, said microparticle or nanoparticle, when assembled, including apolipoprotein from the mammalian subject. In some instances, synthesizing the microparticle or nanoparticle comprises administering at least the following components to a mammalian subject: an organic or an inorganic nanoparticle core; at least one compound having the structure of Formula I; and at least one apolipoprotein; wherein assembly of the components into the microparticle or nanoparticle occurs in vivo in the mammalian subject.

Further provided are methods for reducing, inducing, or increasing ferroptosis in a cell, comprising contacting the cell with an effective amount of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein. In various aspects, provided are uses of a compound or salt disclosed herein (e.g., a compound of Formula I or as shown in Table A, or a pharmaceutically acceptable salt thereof), and/or microparticles or nanoparticles disclosed herein, and/or a pharmaceutical composition disclosed herein. In some instances, the cell is a cancerous cell. In some instances, reducing, inducing, or increasing ferroptosis in a cell treats or prevents a disease or disorder in a subject. In some instances, the disease or disorder is cancer, e.g., ovarian cancer.

Embodiments described herein as methods also can be described as “uses,” and all such uses are contemplated as embodiments. Likewise, compositions described herein as having a “use” can alternatively be described as processes or methods of using, which are contemplated as embodiments. By way of example, aspects described as methods of treatment have, as alternative embodiments, uses for treatment and uses for manufacture of a medicament for treatment. Products-for-use represent a further related embodiment for describing embodiments for using compounds, particles, and compositions described herein.

The headings herein are for the convenience of the reader and not intended to be limiting. Additional aspects, embodiments, and variations will be apparent from the Detailed Description and/or claims.

The Applicant does not intend to claim subject matter described in the prior-art work of others. Should statutory prior art within the scope of a claim be brought to the attention of the Applicant by a Patent Office or other entity or individual, the Applicant reserves the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows the synthesis of Au⁰-dissolved HDL NPs. This material was synthesized using a 5 nm gold nanoparticle (AuNP) as a scaffold. Self-assembly of apoA-I and two phospholipids (PDP-PE and DPPC) around this scaffold yielded the Au⁰-HDL NP starting material. To dissolve the Au core, KCN is added to the Au⁰-HDL NPs, resulting in the gold in the core going from a Au⁽⁰⁾ to Au^{(I)} state after incubation. The desired Au⁰-dissolved HDL NPs are discoidal in shape (TEM) and no longer have the characteristic peak at 520 nm in its UV-vis spectrum, demonstrating the gold is dissolved (HDL NP: top trace; Au⁰-dissolved HDL NP, bottom trace). The Au⁰-dissolved HDL NPs are slightly larger in size than the parent Au⁰-HDL NP starting material and have a negative charge.

FIG. 2 shows that Au⁰-dissolved HDL NPs kill clear cell renal cell carcinoma (ccRCCa) cells. 786-0 ccRCCa cells were treated with aliquots of 390 pM solutions of nanoparticles or controls and viability assessed by MTS assay. HDL NPs, Au⁰-dissolved HDL NPs, and Au⁰-dissolved HDL NPs dialyzed with PBS resulted in a significant decrease in cell viability (** p<0.0001). The [water+KCN] control showed a slight decrease in cell viability (* p=0.01), but when this control was applied after PBS dialysis, there was not a significant decrease in cell viability. These data demonstrate that the Au⁰-dissolved HDL NPs, specifically the remainder PLS-Au complexes in its composition and not the KCN, led to the decrease in the viability of ccRCCa cells.

FIG. 3 shows the synthesis and characterization of DPPTE-Au-salt-containing HDL NPs. First, DPPTE-containing HDL NPs were synthesized using a 5 nm AuNP core as a scaffold with apoA-I protein and DPPTE phospholipids. The resulting materials were further incubated with Et₃PAuCl under basic conditions to produce the desired DPPTE-Au-containing HDL NPs. The resulting particles were larger in size than DPPTE HDL NPs and HDL NPs as evidenced by dynamic light scattering (DLS) and had a more negative charge as evidenced by zeta potential, consistent with the larger head group in the DPPTE Gold Salt HDL NPs and more negative DPPTE phospholipid vs DPPC in HDL NPs, respectively.

FIG. 4 shows that DPPTE-Au-salt-containing HDL NPs kill renal cell carcinoma cells better than HDL NPs. DPPTE-Au-salt-containing HDL NPs inhibited 786-0 ccRCCa cell viability more than DPPTE-containing HDL NPs or the parent Au⁰-HDL NPs, as seen by lower IC₅₀ values produced from MTS assay. In addition, as the Et₃PAuCl/DPPTE ratio in the HDL NP composition increased, the IC₅₀ value decreased, demonstrating the potency of the in-situ-synthesized lipid gold salt DPPTE-AuPEt₃. The concentration of NPs was calculated using Beer's law and UV-vis spectroscopy, where the 5 nm AuNPs have a characteristic absorption at at λₘₐₓ=520 nm (ε=9.696×10⁶ M⁻¹ cm⁻¹).

FIG. 5 shows the synthesis of DPPTE-AuPEt₃-incorporated HDL NPs. DPPTE-AuPEt₃-inc HDL NP were synthesized using a 5 nm gold nanoparticle (AuNP) as a scaffold. Self-assembly of the protein apolipoprotein A-I and two phospholipids (PDP-PE and DPPTE-AuPEt₃) yielded the desired material.

FIG. 6 shows the synthesis of CholS-AuPEt₃-incorporated HDL NPs. CholS-AuPEt₃-incorporated HDL NPs were synthesized using a 5 nm gold nanoparticle (AuNP) as a scaffold. Self-assembly of the protein apolipoprotein A-I, two phospholipids (PDP-PE and DPPC), and cholS-AuPEt₃ yielded the desired material.

FIG. 7 shows the synthesis of Auranofin-incorporated HDL NPs. Auranofin-incorporated HDL NPs were synthesized using a 5 nm gold nanoparticle (AuNP) as a scaffold. Self-assembly of the protein apolipoprotein A-I, two phospholipids (PDP-PE and DPPC), and auranofin yielded the desired material.

FIG. 8 shows characterization data for DPPTE-AuPEt₃-, CholS-AuPEt₃-, and Auranofin-incorporated HDL NPs. The DPPTE-AuPEt₃-inc HDL NPs, CholS-AuPEt₃-inc HDL NPs, and Auranofin-inc HDL NP had similar UV-vis spectra as compared to Au⁰-HDL NPs with a signature 5 nm AuNP absorbance at 520 nm. The sizes of all four nanoparticles were similar (~10-11 nm) and all had a negative charge (−40 to −60 mV) from dynamic light scattering (DLS) and zeta potential measurements, respectively.

FIG. 9 shows that LS-AuPEt₃-incorporated Au⁰-HDL NPs inhibit cell viability more than Au⁰-HDL NPs or Auranofin in ovarian cancer cells. ES-2 clear cell ovarian cancer cells were incubated with increasing doses of nanoparticles or controls and viability assessed by MTS assay after 72 h. DPPTE-AuPEt₃-inc HDL NPs and CholS-AuPEt₃-inc HDL NPs killed cancer cells more potently than HDL NPs or auranofin as demonstrated by decreased IC₅₀ values.

FIG. 10 shows that LS-AuPEt₃-incorporated Au⁰-HDL NPs inhibit inflammation more than Au⁰-HDL NPs or Auranofin in human macrophage cells. THP1-Dual macrophage cells were treated with LPS to induce NFκB activity assayed by a colorimetric QUANTIBlue assay as a read out of inflammation. Treatment with DPPTE-AuPEt₃- or CholS-AuPEt₃-inc HDL NPs significantly inhibited LPS-induced NFκB activity more than auranofin, the free LS-AuPEt₃ gold salts, the Au⁰-HDL NPs, or a liposome material that incorporated DPPTE-AuPEt₃.

FIGS. 11, 12, and 13 show the synthesis and characterization of DPPTE-AuPEt₃- and CholS-AuPEt₃-inc oc-HDL NPs. In a typical experiment, the organic core scaffold PE-S₄ and the phospholipids (DOPC, DPPTE-AuPEt₃) were dissolved in dichloromethane (CH₂Cl₂) and added to aqueous solutions of apoA-I, to reach a final mixture comprising 3/1 water/CH₂Cl₂ and a final ratio of components 300/2/1 lipids/apoA-I/PE-S₄. The nanoparticles formed spontaneously as the CH₂Cl₂ phase evaporates. Following purification by centrifugation and dialysis, the resultant materials were assayed for their size (by dynamic light scattering) and zeta potential. The synthesis from a 225/75, 250/50 and 270/30 DOPC/DPPTE-AuPEt₃ lipid mixtures yielded DPPTE-AuPEt₃-incorporated oc-HDL NPs that are comparable in size to natural, spherical mature HDLs. In the case of the cholS-AuPEt₃-inc oc-HDL NPs, cholS-AuPEt₃/PE-S₄ ratios of 25/1 and 50/1 yielded cholS-AuPEt₃-inc oc-HDL NPs comparable in size and charge to natural, spherical mature HDLs.

FIG. 14 shows that DPPTE-AuPEt₃-incorporated oc-HDL NPs potently induce cell death in SR-B1-positive Ramos and SUDHL4 cells. Ramos cells (SR-B1-positive Burkitt's lymphoma cell line), SUDHL4 cells (SR-B1-positive germinal center diffuse large B cell lymphoma cell line) and Jurkat (SR-B1-negative T cell lymphoma cell line) were plated at 16,000 cells/well in a 96-well plate. DPPTE-AuPEt₃-incorporated oc-HDL NPs induced cell death regardless of the DPPTE-AuPEt₃/DOPC ratio used to generate the materials in the SR-B1 positive Ramos and SUDHL4 cell lines. SR-B1-negative Jurkat cells were significantly more resistant to the DPPTE-AuPEt₃-inc oc-HDL NPs. Incorporation of thiocholesterol-AuPEt₃ into the oc-HDL NPs led to some cell death in the Ramos and SUDHL4 cell lines but not the Jurkat cell line. Control oc-HDL NPs, synthesized with unmodified DPPTE in place of the DPPTE-AuPEt₃ phospholipid, did not induce cell death in any of the cell line analyzed here, demonstrating that cell death is being induced by the DPPTE-AuPEt₃ phospholipid rather than the presence of the thiol-containing DPPTE. The molar quantity of oc-HDL NPs was estimated by measuring the molarity of apoA-I in each mixture, followed by dividing by 2.23, the previously derived apoA-I/NP ratio for this synthesis process.

FIG. 15 shows that DPPTE-AuPEt₃- and cholS-AuPEt₃-inc oc-HDL NPs inhibit inflammation in human macrophage cells. THP1-Dual macrophage cells were treated with LPS to induce NFκB activity assayed by a colorimetric QUANTIBlue assay as a read out of inflammation. Treatment with DPPTE-AuPEt₃-inc oc-HDL NPs (225/75 DOPC/DPPTE-AuPEt₃), control DOPC/DPPTE oc-HDL NPs, and Au-HDL NPs all significantly reduced the QUANTIBlue signal. The cholS-AuPEt₃-inc oc-HDL NPs were slightly less effective at inhibiting LPS-induced NFκB signaling.

FIG. 16 shows the synthesis and characterization of aura-inc oc-HDL NPs. In a typical experiment, the organic core scaffold PE-S₄, auranofin, and DOPC were dissolved in dichloromethane (CH₂Cl₂) and added to aqueous solutions of apoA-I, to reach a final mixture comprising 3/1 water/CH₂Cl₂ and a final ratio of components 300/2/1 lipids/apoA-I/PE-S₄. The nanoparticles formed spontaneously as the CH₂Cl₂ phase evaporated. Following purification by centrifugation and dialysis, the resultant materials were assayed for their size (by dynamic light scattering) and zeta potential. A ratio of 50/1 auranofin/PE-S₄ was used to generate the aura-inc oc-HDL NPs

FIG. 17 shows that Aura-inc oc-HDL NPs induce cell death in SR-B1 positive Ramos and SUDHL4 cells. Ramos cells (SR-B1 positive Burkitt's lymphoma cell line), SUDHL4 cells (SR-B1-positive germinal center diffuse large B cell lymphoma cell line), and Jurkat (SR-B1-negative T cell lymphoma cell line) were plated at 16,000 cells/well in a 96-well plate. Aura-inc oc-HDL NPs induced cell death in the SR-B1-positive Ramos and SUDHL4 cell lines. SR-B1-negative Jurkat cells were significantly more resistant to the aura-inc oc-HDL NPs. Control oc-HDL NPs, synthesized without auranofin, did not induce cell death in any of the cell line analyzed here, demonstrating that cell death is being induced by the auranofin that is directly incorporated into the aura-inc oc-HDL NPs. The molar quantity of oc-HDL NPs was estimated by measuring the molarity of apoA-I in each mixture, followed by dividing by 2.23, the previously derived apoA-I/NP ratio for this synthesis process.

FIG. 18 shows the survival trend for Mantle Cell Lymphoma (MCL) cells (top: REC cells, bottom: MINO cells) 120 hours after treatment with HDL-NPs as disclosed herein.

FIG. 19 shows oxidative stress in Mantle Cell Lymphoma (MCL) cells after treatment with HDL-NPs as disclosed herein, as measured by mean fluorescence intensity (MFI): top left: REC cells, stained with CellROX, top right: REC cells, stained with C11 BODIPY; bottom left: MINO cells, stained with CellROX, bottom right: MINO cells, stained with C11 BODIPY.

FIG. 20 shows that Au-HDL NPs reduce GPX4 expression in MCL cell lines. 1: PBS, 2: Au-HDL NP, 3: DPPTE-AuPEt3 Au-HDL NP, 4: Auranofin Au-HDL NP, 5: ocHDL NP, 6: DPPTE-AuPEt3 ocHDL NP, 7: Auranofin ocHDL NP (both gels).

FIG. 21 shows the results of a cell viability assay in ES2 ovarian cancer cells treated with HDL-NPs disclosed herein.

FIG. 22 shows the results of a cell viability assay in ES2 ovarian cancer cells treated with gold HDL-NPs disclosed herein.

FIG. 23 shows the results of a cell viability assay in ES2 ovarian cancer cells treated with gold salts disclosed herein.

FIG. 24 shows the results of a cell viability assay in TOV-21G ovarian cancer cells treated with gold HDL-NPs disclosed herein.

FIG. 25 shows the results of a cell viability assay in TOV-21G ovarian cancer cells treated with gold salts disclosed herein.

FIG. 26 shows the results of a cell viability assay in OVCAR5 WT (platinum-sensitive) ovarian cancer cells treated with gold HDL-NPs, gold salts, or liposomes disclosed herein (gold HDL-NPs—top, gold salts alone—middle, and liposomes containing DPPTE alone, or DPPTE-AuPEt₃—bottom).

FIG. 27 shows the results of a cell viability assay in OVCAR5 PtR (platinum-resistant) ovarian cancer cells treated with gold HDL-NPs, gold salts, or liposomes disclosed herein (gold HDL-NPs—top, gold salts alone—middle, and liposomes containing DPPTE alone, or DPPTE-AuPEt₃—bottom).

FIG. 28 shows the results of an oxidized lipid staining assay in OVCAR5 PtR (platinum-resistant) ovarian cancer cells treated with gold HDL-NPs, gold salts, or liposomes disclosed herein.

FIG. 29 shows that OVCAR5 WT and PtR ovarian cancer cells treated with Au-Salt HDL NPs, gold salts, or liposomes disclosed herein exhibit decreased GPX4 protein expression.

FIG. 30 shows the survival trend for prostate cancer cells (top: CWR-R1 cells, middle: LNCaP cells, bottom: 22rv1 cells) 72 hours after treatment with HDL-NPs as disclosed herein.

FIG. 31 shows that CWR-R1 prostate cancer cells treated with Au-Salt HDL NPs disclosed herein and ionizing radiation (IR) exhibit decreased GPX4 protein expression.

FIG. 32 shows that DPPTE-AuPEt3/PDPPE HDL NPs Sensitize CWR-R1 cells to Ionizing Radiation (IR). Top: representative images (n=3) from stained clonogenic assays of CWR-R1 cells treated with PBS or DPPTE-AuPEt3 HDL NPs at increasing doses of ionizing radiation (IR). Middle: surviving fraction of CWR-R1 cells treated with DPPTE-AuPEt3/PDPPE HDL NPs compared to PBS control at different doses of ionizing radiation (IR). Bottom: CWR-R1 cells treated with DPPTE-AuPEt3/PDPPE HDL NPs exhibit enhanced expression of p21 and decreased GPX4 protein in the presence of IR compared to cells treated with PBS control.

FIG. 33 shows that DPPTE-AuPEt3/PDPPE HDL NPs Sensitize 22rv1 cells to Ionizing Radiation (IR). Top: representative images (n=3) from stained clonogenic assays of 22rv1 cells treated with PBS or DPPTE-AuPEt3 HDL NPs at increasing doses of ionizing radiation (IR). Bottom: 22rv1 cells treated with DPPTE-AuPEt3/PDPPE HDL NPs exhibit enhanced expression of p21 and decreased GPX4 protein in the presence of IR compared to cells treated with PBS control.

### DETAILED DESCRIPTION

Gold salts have been used to treat inflammation/rheumatoid arthritis for over 70 years. There are several gold salts that are FDA-approved due to their efficacy and the fact that the side-effect profile is usually mild and generally well-tolerated by patients. However, these therapies are non-targeted and therefore must be systemically implemented at high concentrations, which results in side effects.

To improve the therapeutic efficacy and further reduce side effects of gold salt therapy, it was desirable to synthesize new lipid molecules containing gold salts (LS-AuPEt₃). Exemplary molecules described herein are DPPTE-AuPEt₃ and cholS-AuPEt₃ (Compounds 1 and 2 disclosed herein, respectively). DPPTE is also known as diacyl phospholipid, the 1,2-dipalmitoyl-sn-glycero-3-phosphothioethanol, and cholS is also known as cholesterol sulfate. These molecules were used as building blocks to make lipid nanoparticles, including nanoparticles that are highly targeted and called high-density lipoprotein-like nanoparticles (HDL NPs). Data show that these novel materials, exemplified by DPPTE-AuPEt₃-incorporated HDL NPs and cholS-AuPEt₃-incorporated HDL NPs, kill cancer cells at low nanomolar (nM) concentration and potently reduce inflammation at low nM concentrations by reducing NF-κB activity, as demonstrated in a human macrophage cell line. As such, these molecules and their associated lipid nanoparticle therapies are contemplated as useful therapeutics for several human diseases.

The LN and SLN formulations, including the HDL NPs, with the LS-AuPEt₃, whether directly or indirectly incorporated, have therapeutic value based upon the targeted delivery of the HDL NP and its LS-AuPEt₃ cargo. As shown and discussed herein, these compounds/formulations may find use as therapies for some of the most devastating diseases known to humans, including inflammatory diseases (e.g., cardiovascular disease) and cancer, and as new antiparasitic/antimicrobial agents. Furthermore, without intending to be bound by any particular theory, because of the potential targeted nature of these new compounds/formulations, it is contemplated that they will have fewer systemic side effects than other technologies/drugs, including FDA-approved gold salts, that are not targeted to specific diseased cells when administered to patients.

Provided herein are compounds, or pharmaceutically acceptable salt thereof, having the structure of Formula I:

R¹—S—Au—P(R²)₃ (I),
wherein
   R¹ is a steroidal moiety or a lipid moiety; and
   each R² is independently C₁₋₆ alkyl or C₆₋₁₀ aryl. In some cases, the steroidal moiety comprises a sterol derivative. In some cases, the lipid moiety comprises a neutral lipid or a phospholipid derivative. In some cases, R¹ has the structure:

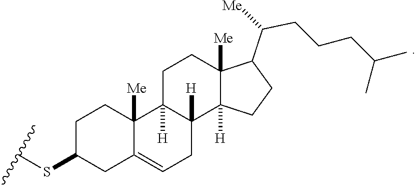

In some cases, R¹ has the structure:

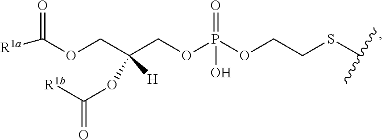

wherein R^{1a} and R^{1b} are each independently C₄₋₂₂ alkyl or C₄₋₂₂ alkenyl, and wherein the C₄₋₂₂ alkenyl is optionally mono- or poly-unsaturated. In some cases, R^{1a} and R^{1b} are each independently C₁₂-C₁₈-alkyl. In some cases, R¹ has the structure:

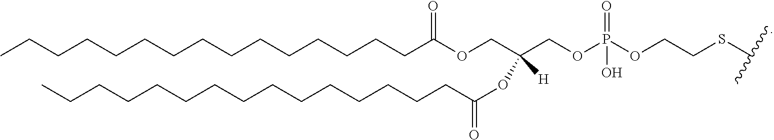 [SMILES: *SCCOP(=O)(O)OC[C@@H](COC(=O)CCCCCCCCCCCCCCC)OC(=O)CCCCCCCCCCCCCCC]

In some cases, each R² is independently C₁₋₃ alkyl. In some cases, at least one R² is ethyl. In some cases, each R² is ethyl.

As used herein, the term “steroidal moiety” refers to a structure derived from a steroid or a steroid derivative. Nonlimiting examples of steroidal moieties include cholesterol moieties (i.e., moieties derived from the structure of cholesterol).

As used herein, the term “lipid moiety” refers to a structure derived from a lipid or a lipid derivative.

As used herein, the term “alkyl” means a saturated straight or branched chain hydrocarbon. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 1-6, 2-6, 1-5, 2-6, 1-4, 2-5, 1, 2, 3, 4, 5, and 6 carbon atoms). Specific examples include, but are not limited to, methyl, ethyl, isopropyl, n-propyl, sec-butyl, and t-butyl. Unless otherwise specified, an alkyl group can be an unsubstituted alkyl group or a substituted alkyl group.

As used herein, the term “alkenyl” refers to refers to an unsaturated straight or branched chain hydrocarbon analogous in length and possible substitution to an alkyl group described above, but that contains at least one double bond. For example, the term “alkenyl” includes straight chain alkenyl groups (e.g., ethenyl, propenyl, butenyl, pentenyl, hexenyl), and branched alkenyl groups. For example, a straight chain or branched alkenyl group can have six or fewer carbon atoms in its backbone (e.g., C₂-C₆ for straight chain, C₃-C₆ for branched chain). The term “C₂-C₆” includes chains having a number of carbon atoms encompassing the entire range (e.g., 2 to 6 carbon atoms), as well as all subgroups (e.g., 2-6, 2-5, 2-4, 3-6, 2, 3, 4, 5, and 6 carbon atoms). Unless otherwise indicated, an alkenyl group can be an unsubstituted alkenyl group or a substituted alkenyl group.

As used herein, the term “aryl” refers to aromatic ring groups having only carbon ring atoms (typically six to ten) and include monocyclic aromatic rings such as phenyl and fused polycyclic aromatic ring systems in which two or more carbocyclic aromatic rings are fused to one another. In some embodiments, aryl is phenyl. An aryl ring is unsubstituted or substituted as described herein.

As used herein, the term “substituted,” when used to modify a chemical functional group, refers to the replacement of at least one hydrogen radical on the functional group with a substituent. Unless otherwise specified for a particular moiety, substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

Also provided are compounds having a structure as shown in Table A, or a pharmaceutically acceptable salt thereof:

TABLE A

| Compound | |
|---|---|
| No. | Structure |
| 1 | 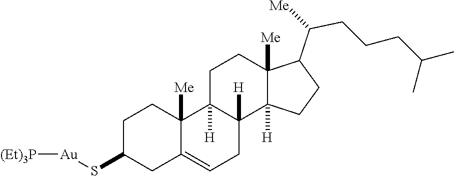 |
| 2 | 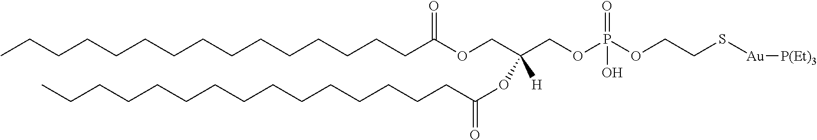 |

Also provided herein are microparticles and nanoparticles comprising a compound as disclosed herein (e.g., a compound of Formula I or a compound of Table A) and a lipid nanoparticle (LN) or a synthetic lipoprotein nanoparticle (SLN). In some cases, the synthetic lipoprotein nanoparticle (SLN) is a high-density lipoprotein-like nanoparticle (HDL-NP) comprising:
(a) an organic or an inorganic nanoparticle core;
(b) a shell surrounding and/or attached to the core wherein the shell comprises one or more apolipoproteins and one or more phospholipids; and
(c) wherein the compound or salt disclosed herein (e.g., a compound of Formula I or a compound of Table A) is associated with one or more of the core or shell. The HDL-NPs of the instant disclosure, can interact with various lipoprotein receptors typically overexpressed in cancer (e.g., scavenger receptor type B1), which can aid in targeting the nanoparticles and the drug to malignant cells. Furthermore, the HDL-NP can be configured (e.g., constructed, modified) to have different hydrophobicity values. For example, without limitation, the quantity and type of the components making up the HDL-NP shell (e.g., apolipoproteins, phospholipids, and/or neutral lipids) can be varied to modulate the particle hydrophobicity. Successful particle fabrication can use inorganic or organic core scaffolds. Specifically, a tetrahedral small molecule-phospholipid hybrid, called PL₄ is contemplated as an exemplary organic core comprising a dendritic scaffold.

The HDL-like nanoparticles of the present disclosure mimic HDL species using lipid-conjugated inorganic or organic cores. The core design motif constrains and orients phospholipid geometry to facilitate the assembly of nanoparticles that are, in some embodiments, approximately 10 nm in diameter and resemble human HDLs in their size, shape, surface chemistry, composition and protein secondary structure. The HDL-like nanoparticles mimic the structure of native HDL with respect to size (~10 nm), surface chemistry (−20 mV zeta potential), and HDL protein secondary structure as determined by circular dichroism. Synthetic HDL-NPs have demonstrated promise as delivery vehicles for therapeutics in applications such as the treatment of an inflammatory disease, an infectious disease, or cancer, among other indications.

Herein, the synthesis of HDL mimetic nanoparticles using lipidated core scaffolds (HDL NPs) is accomplished in a two-step process: first, the core scaffolds are synthesized and purified; second, the particle is fabricated via supramolecular assembly of the core scaffold, free phospholipids, and the HDL-defining protein, apolipoprotein AI (apoA-I).

In some embodiments, the HDL-NP further comprises an apolipoprotein. In some embodiments, the apolipoprotein is apolipoprotein A-I (apoA-I), apolipoprotein A-II (apoA-II), or apolipoprotein E (apo E). In some embodiments, the apolipoprotein is apolipoprotein A-I (apoA-I). In some embodiments, the apolipoproteins have the amino acid sequence of the apolipoproteins proteins from the mammalian species to be treated. For example, human apolipoprotein sequences are selected for nanoparticles or microparticles for use in human therapy.

The shell may be formed, at least in part, of one or more components, such as a plurality of lipids, which may optionally associate with one another and/or with surface of the core. For example, components (e.g., shell, lipid shell) may be associated with the core by being covalently or non-covalently attached to the core, physiosorbed, chemisorbed, or attached to the core through ionic interactions, hydrophobic and/or hydrophilic interactions, electrostatic interactions, van der Waals interactions, or combinations thereof. In some embodiments, the shell is non-covalently attached to the core. In some embodiments, the shell is attached to the core by hydrophobic interactions.

In some embodiments, one or more therapeutic agents (e.g., a compound of Formula I or a compound of Table A) are associated (e.g., by any of the means described herein) with the core. In some embodiments, the one or more therapeutic agents (e.g., a compound of Formula I or a compound of Table A) are associated (e.g., by any of the means described herein) with the shell. In some embodiments, the one or more therapeutic agents (e.g., a compound of Formula I or a compound of Table A) are associated (e.g., by any of the means described herein) to the core and the shell. In some embodiments, as described elsewhere herein, the HDL-NP comprises an apolipoprotein; in some such embodiments, the one or more therapeutic agents (e.g., a compound of Formula I or a compound of Table A) are associated with an apolipoprotein. In some embodiments, the one or more therapeutic agents (e.g., a compound of Formula I or a compound of Table A) are associated to the core and an apolipoprotein. In some embodiments, the one or more therapeutic agents (e.g., a compound of Formula I or a compound of Table A) are associated to the core, shell, and an apolipoprotein. In some embodiments, the one or more therapeutic agents (e.g., a compound of Formula I or a compound of Table A) are associated to the core, shell, and an apolipoprotein. In some embodiments, as described elsewhere herein, the HDL-NP comprises additional components; in some such embodiments, the one or more therapeutic agents (e.g., a compound of Formula I or a compound of Table A) are associated with any additional component. In some embodiments, the one or more therapeutic agents (e.g., a compound of Formula I or a compound of Table A) are associated to the outer layer of a shell. In some embodiments, the one or more therapeutic agents (e.g., a compound of Formula I or a compound of Table A) are associated to the inner layer of a shell. In some embodiments, the attachment is by hydrophobic interactions. In some embodiments, the attachment is a non-covalent attachment.

A microparticle or nanoparticle as disclosed herein may comprise any number of compounds disclosed herein (e.g., a compound of Formula I or a compound of Table A) and optionally therapeutic agents as can be attached to the microparticle or nanoparticle. In some embodiments, a microparticle or nanoparticle comprises at least 5 units or molecules of compounds disclosed herein (e.g., a compound of Formula I or a compound of Table A) and optionally therapeutic agents per core (e.g., a ratio of 5:1 agents:core, e.g., 5:1 agents:PLA₄ core). In some embodiments, a microparticle or nanoparticle comprises at least 10, at least 20, at least 40, at least 50, at least 75, at least 100, at least 150, at least 200, at least 250, at least 300, at least 400, at least 500, at least 600, at least 700, at least 800, at least 900, at least 1,000, at least 1,100, at least 1,200, at least 1,300, at least 1,400, at least 1,500, or at least 2,000 units or molecules of compounds disclosed herein (e.g., a compound of Formula I or a compound of Table A) and optionally therapeutic agents per core (e.g., a ratio of 1000:1 agents:core, e.g., 1000:1 F7:PLA₄ core). In some embodiments, a microparticle or nanoparticle comprises a ratio of at least 5:1, at least 10:1, at least 20:1, at least 40:1, at least 50:1, at least 75:1, at least 100:1, at least 150:1, at least 200:1, at least 250:1, at least 300:1, at least 400:1, at least 500:1, at least 600:1, at least 700:1, at least 800:1, at least 900:1, at least 1,000:1, at least 1,100:1, at least 1,200:1, at least 1,300:1, at least 1,400:1, at least 1,500:1, or at least 2,000:1 units or molecules of compounds disclosed herein (e.g., a compound of Formula I or a compound of Table A) and optionally therapeutic agents relative to core. In some embodiments, a microparticle or nanoparticle comprises a ratio of at least 5:1, at least 10:1, at least 20:1, at least 40:1, at least 50:1, at least 75:1, at least 100:1, at least 150:1, at least 200:1, at least 250:1, at least 300:1, at least 400:1, at least 500:1, at least 600:1, at least 700:1, at least 800:1, at least 900:1, at least 1,000:1, at least 1,100:1, at least 1,200:1, at least 1,300:1, at least 1,400:1, at least 1,500:1, or at least 2,000:1 units or molecules of compounds disclosed herein (e.g., a compound of Formula I or a compound of Table A) and optionally therapeutic agents relative to PL₄ core.

One or more lipids and/or lipid analogs may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example and without limitation, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, 1,2-dipalmitoyl-sn-glycero-3-phosphoethanolamine-N-[3-(2-pyridyldithio)propionate] (PDP-PE), 1,2-dipalmitoyl-sn-glycero-3-phosphocholine (DPPC), and combinations thereof. In some cases, a shell (e.g., a bilayer) of a structure includes 50-200 natural or synthetic lipids or lipid analogs (e.g., phospholipids). For example, without limitation the shell may include less than about 500, less than about 400, less than about 300, less than about 200, or less than about 100 natural or synthetic lipids or lipid analogs (e.g., phospholipids), e.g., depending on the size of the structure.

In some embodiments, a structure described herein includes one or more neutral lipids. Non-limiting examples of neutral lipids include steryl esters [e.g., cholesteryl ester (CE)], triacylglycerols, and wax esters.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E, and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species, that optionally imparts hydrophobicity to the structure.

In some embodiments, the HDL-NP of the instant disclosure further comprise apolipoprotein. The apolipoprotein can be apolipoprotein A (e.g., apoA-I, apoA-II, apoA-IV, and apoA-V), apolipoprotein B (e.g., apoB48 and apoB100), apolipoprotein C (e.g., apoC-I, apoC-II, apoC-III, and apoC-IV), and apolipoproteins D, E (apo E), and H. Additionally or alternatively, a structure described herein may include one or more peptide analogues of an apolipoprotein, such as one described above. Of course, other proteins (e.g., non-apolipoproteins) can also be included in the nanostructures described herein. In some embodiments, the apolipoprotein is apolipoprotein A-I (apoA-I).

The HDL-NP has an inorganic or an organic core. An “inorganic core” as used herein refers to an inorganic material (e.g., a metallic material), hard-core, having a 3-dimensional structure and charge sufficient to organize and hold a lipid layer in a stable shape. An “organic core” as used herein refers to non-metallic material, soft-core, having a 3-dimensional structure and charge sufficient to organize and hold a lipid layer in a stable shape. In some embodiments, the shape is spherical. A “spherical” shape or structure herein refers to a structure having a round or sphere-like structure. The structure does not need to be perfectly round or an exact sphere, but rather is an approximate sphere shape. A variety of inorganic or organic cores can theoretically be used for particle assembly. For example, the core can be an organic core, such as an organic core comprising a dendritic scaffold. In various embodiments, the dendritic scaffold has one or more lipid-conjugated arms. In some cases, the dendritic scaffold is a hydrophobic phospholipid-conjugated scaffold. In some cases, the dendritic scaffold comprises one or more headgroup-modified phospholipids. In various cases, the dendritic scaffold comprises four headgroup-modified phospholipids (PL₄). Herein, successful particle fabrication using a tetrahedral small molecule-phospholipid hybrid, called PL₄ is described. In some cases, the dendritic scaffold has the structure:

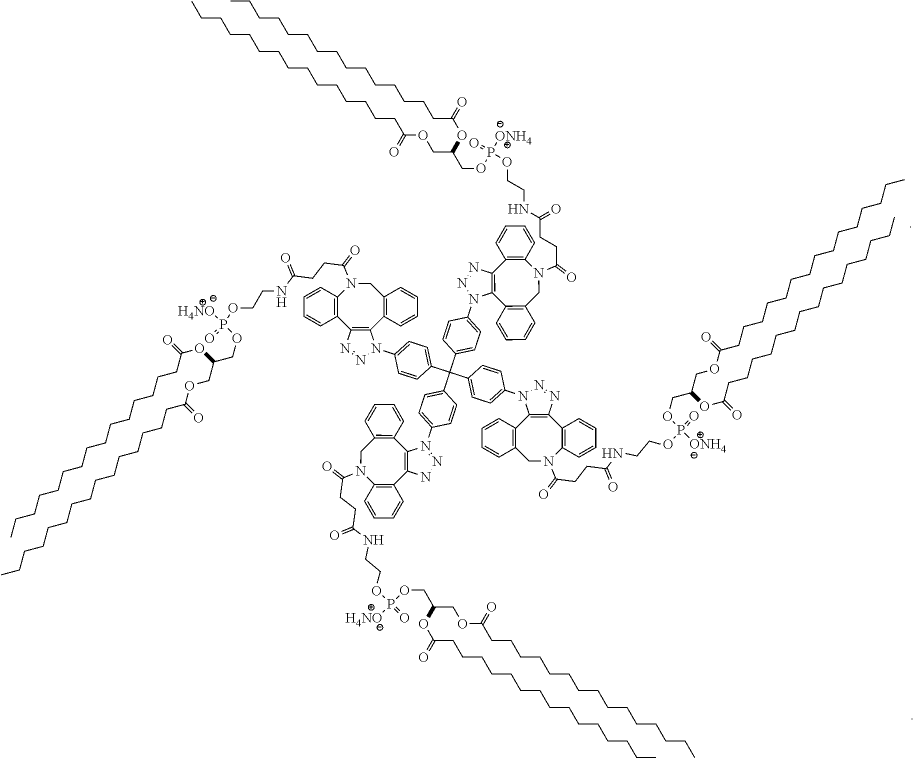

In some cases, the dendritic scaffold comprises four 3-(stearoyloxy)-2,2-bis[(stearoyloxy)methyl]propyl stearate (PE-S₄). In some cases, the dendritic scaffold has the structure:

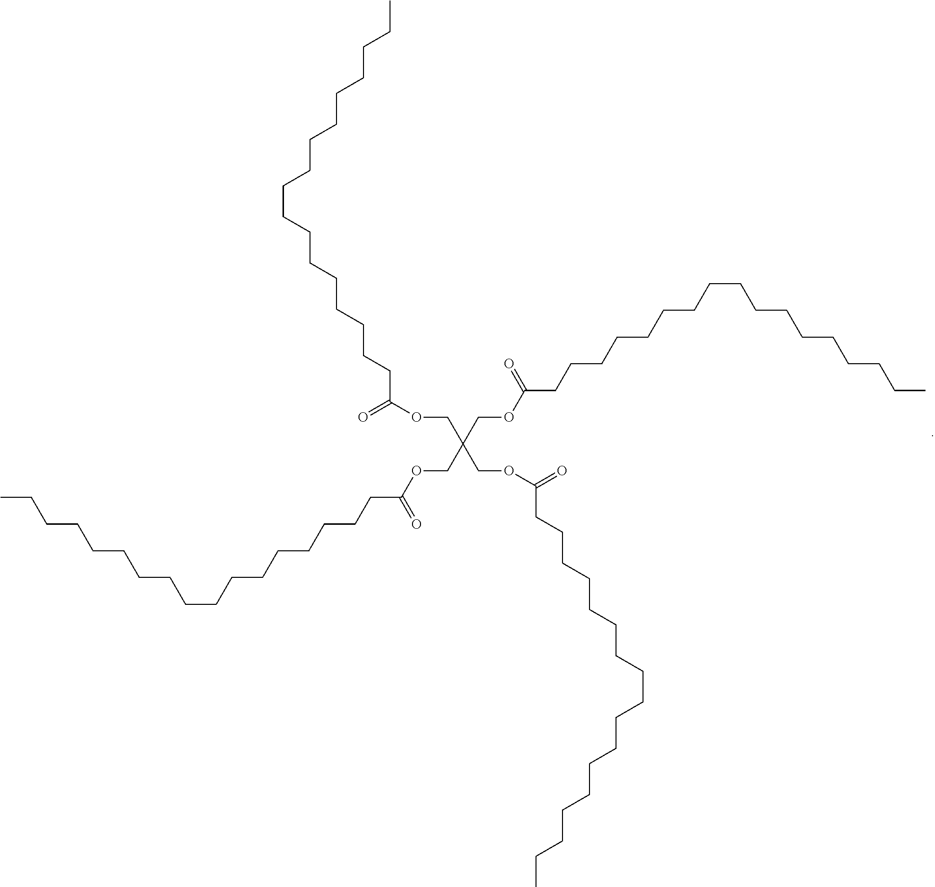 [SMILES: CCCCCCCCCCCCCCCCCC(=O)OCC(COC(=O)CCCCCCCCCCCCCCCCC)(COC(=O)CCCCCCCCCCCCCCCCC)COC(=O)CCCCCCCCCCCCCCCCC]

In some cases, the core is an inorganic nanoparticle core. In some cases, the core is a gold nanoparticle core.

The size, physical properties and functional properties of the HDL-NP of the instant disclosure are similar to that of naturally occurring HDL-NP and distinct from other synthetic HDL-NP. These properties include, for example, spherical shape, surface chemistry, size, hydrodynamic diameter, zeta potential, cholesterol efflux, cholesterol delivery, and therapeutic functions such as suppression of inflammation.

In some embodiments, the HDL-NP of the instant disclosure, can be assessed based on the hydrodynamic diameter. The hydrodynamic diameter of the HDL-NP is similar to that of naturally occurring HDL-NP and distinct from other synthetic HDL-NP. Hydrodynamic diameter assesses the size of a hypothetical hard sphere that diffuses in the same manner as that of the particle being measured and provides an indication of the diffusional properties of the particle that will be indicative of the apparent size of the dynamic hydrated/solvated particle. It may be measured by Dynamic Light Scattering (DLS)

The HDL-NPs of the present disclosure can have a diameter with a largest cross-sectional dimension (or, sometimes, a smallest cross-section dimension) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some embodiments the HDL-NP has a diameter of about 5-30 nm, 5-25 nm, 5-22 nm, 5-20 nm, 5-15 nm, 5-14 nm, 5-13 nm, 5-12 nm, 5-11 nm, 5-10 nm, 8-15 nm, 8-14 nm, 8-13 nm, 8-12 nm, 8-11 nm, 8-10 nm, 10-12 nm, or 10 nm. In some cases, the HDL-NP has a diameter of about 5 nm to about 150 nm. In some cases, the HDL-NP has a diameter of about 5 nm to about 30 nm, about 5 nm to about 20 nm, about 5 nm to about 15 nm, about 5 nm to about 10 nm, about 8 nm to about 13 nm, about 8 nm to about 12 nm, or about 10 nm.

In some embodiments, the shell has a zeta potential closer to human HDL than a synthetic HDL nanoparticle with an inorganic core. In some embodiments, the HDL-NP has a zeta potential closer to human HDL than a synthetic HDL nanoparticle with a gold core. In some embodiments, the HDL-NP has a zeta potential of zeta potential of −16 to −60 mV. All integer subranges of −16 to −60 mV and all integer values defined by the range −16 to −60 mV are specifically contemplated. In some embodiments, the zeta potential of the HDL-NPs is about −20 millivolts (mV). In some embodiments, the zeta potential of the HDL-NPs is selected from a group consisting of −10 mV, −12 mV, −14 mV, −16 mV, −18 mV, −20 mV, −22 mV, −24 mV, −26 mV, and −30 mV. In some embodiments, the zeta potential of the HDL-like nanoparticles is greater than −20 mV. In some embodiments, the zeta potential is less than −20 mV. In some embodiments, the zeta potential is that of human HDL. Zeta potential may be assessed using methods known in the art, including the methods disclosed herein.

In some embodiments, the HDL-NP comprises one or more therapeutic agents.

In some embodiments, the one or more therapeutic agents comprise an anti-inflammatory, anti-oxidants, anti-microbial, anti-viral, anti-cancer agents, or polyunsaturated fatty acids, or a combination thereof. In some embodiments, the one or more therapeutic agents comprise an anti-inflammatory. In some embodiments, the one or more therapeutic agents comprise anti-oxidants. In some embodiments, the one or more therapeutic agents comprise an anti-microbial. In some embodiments, the one or more therapeutic agents comprise an anti-viral. In some embodiments, the one or more therapeutic agents comprise polyunsaturated fatty acids. In some embodiments, the one or more therapeutic agents comprise anti-cancer agents. In some embodiments, the one or more therapeutic agents comprise a chemotherapeutic agent. In some embodiments, the one or more therapeutic agents comprise auranofin, PIK75 (F7) (C₁₆H₁₄BrN₅O₄S·HCl), doxorubicin, vincristine, gemcitabine, paclitaxel, docetaxel, andrographolide, sutent, tamoxifen, vitamin D, vitamin E, or a combination thereof.

The compounds disclosed herein (e.g., a compound of Formula I or compound of Table A), compositions thereof, microparticle or nanoparticle formulations comprising the compounds disclosed herein (e.g., a compound of Formula I or compound of Table A), and methods of the present disclosure can be used in applications including, but not limited to treating or preventing a disease or disorder. In some cases, the disease or disorder is an inflammatory disease, infectious disease, or cancer. In some cases, the disease or disorder is an inflammatory disease. In some cases, the disease or disorder is an infectious disease.

In some cases, the disease or disorder cancer. Accordingly, in some aspects, the disclosure relates to methods of treating or preventing an inflammatory disease, infectious disease, or cancer comprising administering to a subject in need thereof a therapeutically effective amount of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A). Further provided are methods of reducing inflammation comprising administering to a subject in need thereof comprising administering to a subject in need thereof a therapeutically effective amount of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A).

Also provided are methods of inhibiting neoplastic cell growth comprising administering to a subject in need thereof comprising administering to a subject in need thereof a therapeutically effective amount of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A).

Also contemplated are the use of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A) for the treatment or prevention of an inflammatory disease, infectious disease, or cancer.

Also contemplated is the use of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A) or a pharmaceutically acceptable salt thereof in the manufacture of a medicament for the treatment or prevention of an inflammatory disease, infectious disease, or cancer.

Inflammatory diseases are characterized by inflammatory abnormalities, and/or by causal origins in inflammatory processes. Non-limiting examples of inflammatory diseases include psoriasis, psoriatic arthritis, atopic dermatitis, Sjogren's syndrome, multiple sclerosis, amyotrophic lateral sclerosis, neuroinflammation, carditis, myocarditis, angiitis, arteritis, nephritis, neuritis, pleuritis, pneumonitis, arthritis, polyarthritis, optic neuritis, ophthalmitis, meningitis, myalgia, polymyalgia, polymyalgia rheumatica, gastroenteritis, enteritis, esophagitis, Crohn's disease, ulcerative colitis, colitis, proctitis, interstitial cystitis, cystitis, irritable bowel syndrome, dermatitis, mucositis, urethritis, and prostatitis. In some cases, the arthritis is rheumatoid arthritis.

An infectious disease, also known as a transmissible disease or communicable disease, is an illness resulting from an infection. An infection is the invasion of tissues by pathogens, their multiplication, and the reaction of host tissues to the infectious agent and the toxins they produce. In some cases, the infectious disease is a parasitic disease, a bacterial disease, a fungal disease, or a viral disease.

Cancers are generally characterized by unregulated cell growth, formation of malignant tumors, and invasion to nearby parts of the body. Cancers may also spread to more distant parts of the body through the lymphatic system or bloodstream. Cancers may be a result of gene damage due to tobacco use, certain infections, radiation, lack of physical activity, obesity, and/or environmental pollutants. Cancers may also be a result of existing genetic faults within cells to cause diseases due to genetic heredity. Screenings may be used to detect cancers before any noticeable symptoms appear and treatment may be given to those who are at higher risks of developing cancers (e.g., people with a family history of cancers). Examples of screening techniques for cancer include but are not limited to physical examination, blood or urine tests, medical imaging, and/or genetic testing.

Non-limiting examples of cancers include: chronic myeloid leukemia (CML), adult acute myeloid leukemia (AML), acute lymphocytic leukemia (ALL), bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer. Additional non-limiting examples of cancer include Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, soft tissue Ewing's sarcoma, soft tissue sarcoma, synovial sarcoma, malignant lymphoma (reticulum cell sarcoma), multiple myeloma, malignant giant cell tumor chordoma, desmoid-type fibromatosis, fibroblastic sarcoma, gastrointestinal stromal tumors, retroperitoneal sarcoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynaecological sarcoma, Kaposi's sarcoma, peripheral never sheath tumor, Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. In some embodiments, the subject of any one of the methods of the disclosure has cancer. In some embodiments the cancer at least one cancer selected from renal cancer, chronic myeloid leukemia (CML), multiple myeloma (MM), adult acute myeloid leukemia (AML), acute lymphocytic leukemia (ALL), cutaneous T cell lymphoma (CTCL), melanoma, ovarian cancer, breast cancer, gastrointestinal malignancies, and/or brain tumors. In some embodiments, the cancer is renal cancer. In some embodiments, the cancer is cutaneous T cell lymphoma (CTCL). In some embodiments, the cancer is colon cancer. In some cases, the cancer is sarcoma, uterine cancer, cervical cancer, lung cancer, renal cancer, leukemia, lymphoma, multiple myeloma, melanoma, ovarian cancer, breast cancer, gastrointestinal malignancies, brain tumors, prostate cancer, or colon cancer.

The compounds disclosed herein (e.g., a compound of Formula I or compound of Table A) and pharmaceutically acceptable salts thereof, HDL-NPs and/or compositions administered in an effective amount can be administered for prophylactic or therapeutic treatments. As used herein, the term “treating” or “treatment” refers to the application or administration of the HDL-NPs and/or compositions thereof, to a subject who has a disease or disorder (e.g., cancer), a symptom of a disease or disorder (e.g., cancer), or is at risk of a disease or disorder (e.g., cancer), with the purpose to prevent, cure, heal, alleviate, relieve, alter, remedy, ameliorate, improve, or affect the disorder resulting from the disease (e.g., cancer). In prophylactic applications, the HDL-NPs and/or compositions can be administered to a subject (e.g., patient) with a clinically determined predisposition or increased susceptibility to development of a disorder associated a hyperproliferative diseases (e.g., cancer). The HDL-NPs and/or compositions of the present disclosure can be administered to the subject (e.g., patient (e.g., a human)) in an amount sufficient to delay, reduce, or preferably prevent the onset of the clinical disease. In therapeutic applications, HDL-NPs and/or compositions are administered to a subject (e.g., patient (e.g., a human) already suffering from a hyperproliferative diseases (e.g., cancer) and/or other pathological conditions associated with the condition and its complications. An amount adequate to accomplish this purpose is defined as an “effective amount,” “therapeutically effective amount,” or “therapeutically effective dose,” and is an amount of a compound (e.g., HDL-NP and/or composition) sufficient to substantially improve some symptom associated with a disease or a medical condition. A therapeutically effective amount of an agent or composition is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Also contemplated is the use of a compound or salt disclosed herein (e.g., a compound of Formula I, a compound of Table A, or a pharmaceutically acceptable salt of the compound or Formula I or Table A), microparticle or nanoparticle formulations comprising the compounds disclosed herein (e.g., a compound of Formula I or compound of Table A), or the pharmaceutical composition comprising a compound or salt disclosed herein (e.g., a compound of Formula I or compound of Table A) or a microparticle or nanoparticle formulation comprising the compounds disclosed herein (e.g., a compound of Formula I or compound of Table A) or a pharmaceutically acceptable salt thereof in the method for reducing inducing or increasing ferroptosis in a cell. As used herein, the term “ferroptosis” refers to a type of programmed cell death dependent on iron and characterized by the accumulation of lipid peroxides, and is genetically and biochemically distinct from other forms of regulated cell death such as apoptosis. Without wishing to be bound by any particular theory, ferroptosis is initiated by the failure of the glutathione-dependent antioxidant defenses, resulting in unchecked lipid peroxidation and eventual cell death. In some cases, contacting the cell with the compound, salt, microparticle formulation, nanoparticle formulation, or pharmaceutical formulation is performed in vivo. In some cases, inducing or increasing ferroptosis in a cell treats or prevents a disease in a subject, e.g., a disease as disclosed herein. In some cases, the disease is cancer. In some cases, the disease is ovarian cancer. In some cases, the cell is a cancerous cell.

As used herein, a “subject” or a “patient” refers to any mammal (e.g., a human), for example, a mammal that may be susceptible to a disease or bodily condition such as the secondary diseases or conditions disclosed herein. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, a disease or bodily condition associated with cancer, as described herein. In certain embodiments, a subject may be selected for treatment on the basis of a known disease or bodily condition in the subject. In some embodiments, a subject may be selected for treatment on the basis of a suspected disease or bodily condition in the subject.

In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

The term “prevent” reflects the ability of an agent to reduce or delay the incidence of a disease or condition in a population of treated individuals compared to a similar population of untreated controls over a finite period of time. In this respect, successful prevention can be demonstrated in a pre-clinical or clinical trial using a plurality of suspects, or retrospectively from examining disease incidence/onset retrospectively in treated individuals compared to untreated controls.

In some embodiments, the compounds and salts thereof (e.g., a compound of Formula I or compound of Table A) and HDL-NPs of the present disclosure are in a pharmaceutical composition. These “pharmaceutical compositions” or “pharmaceutically acceptable” compositions (also referred to herein simply as “compositions” of the compounds and salts thereof (e.g., a compound of Formula I or compound of Table A) HDL-NPs), may comprise a therapeutically effective amount of one or more of the compounds or salts thereof (e.g., a compound of Formula I or compound of Table A) and/or structures described herein (e.g., HDL-NPs), formulated together with one or more pharmaceutically acceptable carriers, additives, and/or diluents. It should be understood that any compounds and salts thereof (e.g., a compound of Formula I or compound of Table A) and suitable structures described herein can be used in such pharmaceutical compositions, including those described in connection with the figures.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The compounds and salts thereof (e.g., a compound of Formula I or compound of Table A) and structures described herein may be administered by intrathecal, intravenous, intraperitoneal, oral, topical, ocular, intratumoral, or intra-articular administration. In certain embodiments, a structure or pharmaceutical preparation is administered orally. In other embodiments, the compound or salt thereof (e.g., a compound of Formula I or compound of Table A), structure, or pharmaceutical preparation is administered intravenously.

Also provided herein are processes of synthesizing a compound of Formula (I):
R¹—S—Au—P(R²)₃ (I), comprising
admixing a compound of Formula (I′):
R¹—SH (I′) and a compound of Formula (I″):
X—Au—P(R²)₃ (I″) in the presence of a base to form the compound of Formula (I),
wherein
   R¹ is a steroidal moiety or a lipid moiety;
   each R² is independently C₁₋₆ alkyl or C₆₋₁₀ aryl; and
   X is a halogen ion. As used herein, the term “steroidal moiety” refers to a structure derived from a steroid or a steroid derivative. Nonlimiting examples of steroidal moieties include cholesterol moieties (i.e., moieties derived from the structure of cholesterol). As used herein, the term “lipid moiety” refers to a structure derived from a lipid or a lipid derivative. Nonlimiting examples of lipid moieties include phospholipid moieties (i.e., moieties derived from the structure of a phospholipid). In some cases, the steroidal moiety comprises a sterol derivative. In some cases, the lipid moiety comprises a neutral lipid or a phospholipid derivative. In some cases, R¹ has the structure:

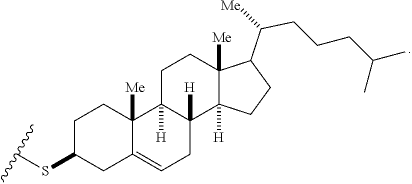

In some cases, R¹ has the structure:

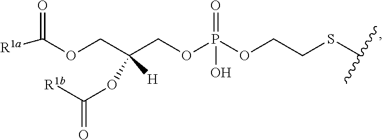

wherein R^{1a} and R^{1b} are each independently C₄₋₂₂ alkyl or C₄₋₂₂ alkenyl, and wherein the C₄₋₂₂ alkenyl is optionally mono- or poly-unsaturated. In some cases, R^{1a} and R^{1b} are each independently C₁₂-C₁₈-alkyl. In some cases, R¹ has the structure:

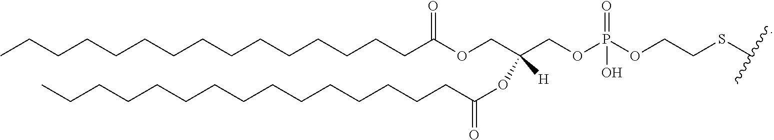 [SMILES: *SCCOP(=O)(O)OC[C@@H](COC(=O)CCCCCCCCCCCCCCC)OC(=O)CCCCCCCCCCCCCCC]

In some cases, each R² is independently C₁₋₃ alkyl. In some cases, at least one R² is ethyl. In some cases, each R² is ethyl. In some cases, the base is an alkali carbonate. In some cases, the base is potassium carbonate. In some cases, X is chloride.

Also provided are processes of synthesizing a microparticle or nanoparticle described herein, comprising:
(i) admixing one or more gold nanoparticles (AuNPs) and one or more apolipoproteins to form one or more Apo-AuNPs;
(ii) incubating the one or more Apo-AuNPs;
(iii) admixing the one or more Apo-AuNPs and a compound of Formula (I′):
R¹—SH (I′) to form one or more R¹S-Apo-AuNPs;
(iv) incubating the one or more R¹S-Apo-AuNPs; and
(v) admixing the one or more R¹S-Apo-AuNPs and a compound of Formula (I″):
X—Au—P(R²)₃ (I″) in the presence of a base to form the microparticle or nanoparticle,
wherein
   X is a halogen ion. In some cases, the one or more apolipoproteins is apolipoprotein A-I (apoA-I) or apolipoprotein E (apo E). In some cases, the one or more apolipoproteins is apolipoprotein A-I (apoA-I). In some cases, X is chloride. In some cases, the base is an alkali carbonate. In some cases, the base is potassium carbonate. In some cases, incubating the one or more Apo-AuNPs occurs at a temperature of about 5 °C to about 35 °C. In some cases, incubating the one or more Apo-AuNPs occurs at a temperature of about 20 °C to about 25 °C. In some cases, incubating the one or more Apo-AuNPs occurs for about 8 to about 18 hours. In some cases, incubating the one or more R¹S-Apo-AuNPs occurs at a temperature of about 5 °C to about 35 °C. In some cases, incubating the one or more R¹S-Apo-AuNPs occurs at a temperature of about 20 °C to about 25 °C. In some cases, incubating the one or more R¹S-Apo-AuNPs occurs for about 8 to about 18 hours. In some cases, the methods further comprise the step of incubating the microparticle or nanoparticle. In some cases, incubating the microparticle or nanoparticle occurs at a temperature of about 5 °C to about 35 °C. In some cases, incubating the microparticle or nanoparticle occurs at a temperature of about 20 °C to about 25 °C. In some cases, incubating the microparticle or nanoparticle occurs for about 8 to about 18 hours.

Also provided are processes of synthesizing a microparticle or nanoparticle described herein, comprising:
(i) admixing one or more gold nanoparticles (AuNPs) and one or more apolipoproteins in a solvent to form one or more Apo-AuNPs;
(ii) incubating the one or more Apo-AuNPs;
(iii) admixing the one or more Apo-AuNPs and one or more phospholipids in an organic solvent to form one or more PL-Apo-AuNPs;
(iv) admixing the one or more PL-Apo-AuNPs and a compound of Formula (I) in the organic solvent; and
(v) removing the organic solvent to form the microparticle or nanoparticle. In some cases, the one or more apolipoproteins is apolipoprotein A-I (apoA-I) or apolipoprotein E (apo-E).

In some cases, the one or more apolipoproteins is apolipoprotein A-I (apoA-I). In some cases, the one or more phospholipids is dissolved in an organic solvent prior to admixing in step (iii). In some cases, the organic solvent is a chlorinated solvent. In some cases, the organic solvent is CH₂Cl₂. In some cases, the one or more phospholipids comprise 1,2-dipalmitoyl-sn-glycero-3-phosphoethanolamine-N-[3-(2-pyridyldithio)propionate] (PDP-PE).

In some cases, incubating the one or more Apo-AuNPs occurs at a temperature of about 5 °C to about 35 °C. In some cases, incubating the one or more Apo-AuNPs occurs at a temperature of about 20 °C to about 25 °C. In some cases, incubating the one or more Apo-AuNPs occurs for about 1 hour.

Also provided are processes of synthesizing a microparticle or nanoparticle disclosed herein, comprising:
(i) admixing one or more gold nanoparticles (AuNPs) and one or more apolipoproteins in a solvent to form one or more Apo-AuNPs;
(ii) incubating the one or more Apo-AuNPs;
(iii) admixing the one or more Apo-AuNPs and one or more phospholipids to form one or more PL-Apo-AuNPs;
(iv) incubating the one or more one or more PL-Apo-AuNPs;
(v) optionally repeating steps (ii) and (iii) to incorporate one or more additional phospholipids into the PL-Apo-AuNPs; and
(iv) admixing the one or more PL-Apo-AuNPs and aqueous KCN to form the microparticle or nanoparticle. In some cases, the one or more apolipoproteins is apolipoprotein A-I (apoA-I) or apolipoprotein E (apo E). In some cases, the one or more apolipoproteins is apolipoprotein A-I (apoA-I). In some cases, the one or more phospholipids is dissolved in an organic solvent prior to admixing in step (iii). In some cases, the organic solvent is a polar organic solvent. In some cases, the organic solvent is ethanol. In some cases, the one or more phospholipids comprise 1,2-dipalmitoyl-sn-glycero-3-phosphoethanolamine-N-[3-(2-pyridyldithio)propionate](PDP-PE). In some cases, incubating the one or more Apo-AuNPs occurs at a temperature of about 5 °C to about 35 °C. In some cases, incubating the one or more Apo-AuNPs occurs at a temperature of about 20 °C to about 25 °C. In some cases, incubating the one or more Apo-AuNPs occurs for about 1 hour. In some cases, incubating the one or more PL-Apo-AuNPs occurs at a temperature of about 5 °C to about 35 °C. In some cases, incubating the one or more PL-Apo-AuNPs occurs at a temperature of about 20 °C to about 25 °C. In some cases, incubating the one or more PL-Apo-AuNPs occurs for about 1 hour. In some cases, the method further comprises the step of incubating the microparticle or nanoparticle. In some cases, incubating the microparticle or nanoparticle occurs at a temperature of about 5 °C to about 35 °C. In some cases, incubating the microparticle or nanoparticle occurs at a temperature of about 20 °C to about 25 °C. In some cases, incubating the microparticle or nanoparticle occurs for about 8 to about 18 hours.

Also provided are processes of synthesizing a microparticle or nanoparticle disclosed herein, comprising admixing a dendritic scaffold, an apolipoprotein, a sterol salt, a phospholipid, and a compound of Formula (I) to form the microparticle or nanoparticle. In some cases, the dendritic scaffold is a pentaerythrityl tetraester. In some cases, the dendritic scaffold is 3-(stearoyloxy)-2,2-bis[(stearoyloxy)methyl]propyl stearate (PE-S₄). In some cases, the apolipoprotein is apolipoprotein A-I (apoA-I) or apolipoprotein E (apo E). In some cases, the apolipoprotein is apolipoprotein A-I (apoA-I). In some cases, the sterol salt is a cholate salt. In some cases, the phospholipid is 1,2-dioleoyl-sn-glycero-3-phosphocholine (DOPC). In some cases, the ratio of the phospholipid and compound of Formula (I) to the dendritic scaffold is about 300:1. In some cases, the method further comprises the step of incubating the microparticle or nanoparticle. In some cases, incubating the microparticle or nanoparticle occurs at a temperature of about 5 °C to about 35 °C. In some cases, incubating the microparticle or nanoparticle occurs at a temperature of about 20 °C to about 25 °C. In some cases, incubating the microparticle or nanoparticle occurs for about 8 to about 18 hours.

Also provided are processes of synthesizing a microparticle or nanoparticle disclosed herein, wherein assembly of the microparticle or nanoparticle occurs in vivo. In some cases, the one or more apolipoproteins are native apolipoproteins. In some cases, the native apolipoproteins are not administered to a subject. In such processes, the particles can also be formed (can form) in vivo if all of the components except for the apolipoproteins, are administered without pre-assembly. Without wishing to be bound by any particular theory, the body of the subject to whom the components are administered will provide the apolipoproteins in situ, and the particles can be formed in vivo. In some cases, a process described herein can be carried out by administering all of the recited components, including the apolipoproteins, and then the particles are made in vivo. In such cases, the final microparticle or nanoparticle constituents can be formed following therapeutic administration, such as microparticle or nanoparticle opsonization of apolipoprotein, including apolipoprotein A-I (apoA-I) or apolipoprotein E (apo E), after administration.

### Routes of Administration of Radiation Therapy

Radiation therapy involves the use of high-energy radiation (e.g., x-rays, gamma rays, or charged particles) to damage and/or kill cancer cells and to shrink tumors. In the methods of the invention, radiation may be delivered to the patient by a machine positioned outside the body (external-beam radiation therapy), by radioactive material placed in the body near cancer (internal radiation therapy, also called brachytherapy), or by radioactive substances administered systemically (e.g., radioactive iodine) that travel through the bloodstream to the cancer. Alternatively, these delivery methods can be used in combination.

Radiation therapies which are suitable for use in the combination treatments described herein, include the use of a) external beam radiation; and b) a radiopharmaceutical agent which comprises a radiation-emitting radioisotope.

External Beam Radiation: External beam radiation therapy for the treatment of cancer uses a radiation source that is external to the patient, typically either a radioisotope, such as Co, Cs, or a high energy x-ray source such as a linear accelerator. The external source produces a collimated beam directed into the patient to the tumor site. External-source radiation therapy avoids some of the problems of internal-source radiation therapy, but it irradiates a significant volume of non-tumorous or healthy tissue in the path of the radiation beam along with the tumorous tissue. The adverse effect of irradiating healthy tissue can be reduced, while maintaining a given dose of radiation in the tumorous tissue, by projecting the external radiation beam into the patient at a variety of angles with the beams converging on the cancer (e.g., tumor) site. The particular volume elements of healthy tissue along the path of the radiation beam change, reducing the total dose to healthy tissue during the entire treatment. The irradiation of healthy tissue also can be reduced by tightly collimating the radiation beam to the general cross section of the cancer (e.g. tumor) taken perpendicular to the axis of the radiation beam.

Radiopharmaceutical Agents: A “radiopharmaceutical agent” refers to a pharmaceutical agent which contains at least one radiation-emitting radioisotope. Radiopharmaceutical agents are routinely used in nuclear medicine for the diagnosis and/or therapy of various diseases. The radiolabeled pharmaceutical agent, for example, a radiolabeled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule. A “metallic radioisotope” is any suitable metallic radioisotope useful in a therapeutic or diagnostic procedure in vivo or in vitro. Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects. It is preferred that the physical half-life of the therapeutic radioisotope be similar to the biological half-life of the radiopharmaceutical at the tumor site. For example, if the half-life of the radioisotope is too short, much of the decay will have occurred before the radiopharmaceutical has reached maximum target/background ratio. On the other hand, too long a half-life would cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope must be long enough to allow adequate time for manufacturing, release, and transportation.

The type of radiation that is suitable for use in the methods of the present invention can vary. For example, radiation can be electromagnetic or particulate in nature. Electromagnetic radiation useful in the practice of this invention includes but is not limited to x-rays and gamma rays. Particulate radiation useful in the practice of this invention includes, but is not limited to, electron beams (beta particles), protons beams, neutron beams, alpha particles, and negative pi mesons. The radiation can be delivered using conventional radiological treatment apparatus and methods, and by intraoperative and stereotactic methods. Additional discussion regarding radiation treatments suitable for use in the practice of this invention can be found throughout Steven A. Leibel et al., Textbook of Radiation Oncology (1998) (publ. W. B. Saunders Company), e.g., in Chapters 13 and 14. Radiation can also be delivered by other methods such as targeted delivery, for example by radioactive “seeds,” or by systemic delivery of targeted radioactive conjugates. J. Padawer et al., Int. J. Radiat. Oncol. Biol. Phys. 7:347-357 (1981). Particularly preferred forms of radiation for use in the combinations disclosed herein are forms of ionizing radiation.

As used herein, the term “ionizing radiation” refers to subatomic particles or electromagnetic waves that have sufficient energy to ionize atoms or molecules by detaching electrons from them. Nonlimiting examples of ionizing radiation include alpha particles, beta particles, and neutrons (for subatomic particle-type ionizing radiation), and gamma rays, X-rays, and the higher energy ultraviolet part of the electromagnetic spectrum (for electromagnetic wave-type ionizing radiation).

For tumor therapy, both α- and β-particle emitters have been investigated. Alpha particles are particularly good cytotoxic agents because they dissipate a large amount of energy within one or two cell diameters. The β-particle emitters have relatively long penetration range (2-12 mm in the tissue) depending on the energy level. The long-range penetration is particularly important for solid tumors that have heterogeneous blood flow and/or receptor expression. The β-particle emitters yield a more homogeneous dose distribution even when they are heterogeneously distributed within the target tissue.

Administration of External Beam Radiation: For administration of external beam radiation, the amount can be at least about 1 Gray (Gy) fractions at least once every other day to a treatment volume. In some embodiments, the radiation is administered in at least about 2 Gray (Gy), about 4 Gy, about 6 Gy, or about 8 Gy fractions at least once per day to a treatment volume. In another particular embodiment, the radiation is administered in at least about 2 Gray (Gy) fractions at least once per day to a treatment volume for five consecutive days per week. In some embodiments, radiation is administered in 10 Gy fractions every other day, three times per week to a treatment volume. In some embodiments, a total of at least about 20 Gy is administered to a patient in need thereof. In some embodiments, at least about 30 Gy is administered to a patient in need thereof. In some embodiments, at least about 40 Gy is administered to a patient in need thereof. Typically, the patient receives external beam therapy four or five times a week. An entire course of treatment usually lasts from one to seven weeks depending on the type of cancer and the goal of treatment. For example, a patient can receive a dose of 2 Gy/day over 30 days.

Administration of Radiopharmaceutical Agent: There are several methods for administration of a radiopharmaceutical agent. For example, the radiopharmaceutical agent can be administered by targeted delivery or by systemic delivery of targeted radioactive conjugates, such as a radiolabeled antibody, a radiolabeled peptide and a Liposome delivery System. See, for example, Ballangrud A. M., et al. Cancer Res., 2001; 61:2008-2014 and Goldenberg, D M J. Nucl. Med., 2002; 43(5):693-713, the contents of which are incorporated by reference herein. In some embodiments, the radiopharmaceutical agent can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. See, for example, Emfietzoglou D, Kostarelos K, SgouroS G. An analytical dosimetry Study for the use of radionuclide-liposome conjugates in internal radiotherapy. J Nucl Med 2001; 42:499-504, the contents of which are incorporated by reference herein. In some embodiments, the radiolabeled pharmaceutical agent can be a radiolabeled peptide. See, for example, Weiner R E, Thakur M L. Radiolabeled peptides in the diagnosis and therapy of oncological diseases. Appl Radiat Isot 2002 November; 57(5):749 63, the contents of which are incorporated by reference herein.

In addition to targeted delivery, brachytherapy can be used to deliver the radiopharmaceutical agent to the target site. Brachytherapy is a technique that puts the radiation sources as close as possible to the tumor site. Often the source is inserted directly into the tumor. The radioactive sources can be in the form of wires, seeds or rods. Generally, cesium, iridium or iodine are used. There are two types of brachytherapy: intercavitary treatment and interstitial treatment. In intracavitary treatment, containers that hold radioactive sources are put in or near the tumor. The sources are put into the body cavities. In interstitial treatment the radioactive sources alone are put into the tumor. These radioactive sources can stay in the patient permanently. Most often, the radioactive sources are removed from the patient after several days. The amount of radiation necessary can be determined by one of skill in the art based on known doses for a particular type of cancer. See, for example, Cancer Medicine 5″ ed., Edited by R. C. Bast et al., July 2000, B C Decker, the entire content of which is hereby incorporated by reference.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Materials and Instrumentation

Unless otherwise stated, all reagents and reagent-grade solvents were purchased from common commercial sources and used as received. Thiocholesterol, auranofin, 3-(Stearoyloxy)-2,2-bis[(stearoyloxy)methyl]propyl stearate (PE-S₄) and poly(ethylene glycol) sorbitol hexaoleate (Sorb(PEG)₁-O₆) cores were acquired from Sigma-Aldrich (Milwaukee, WI). 1,2-Dipalmitoyl-sn-glycero-3-phosphothioethanol sodium salt (16:0 Ptd thioethanol, aka DPPTE), 1,2-dipalmitoyl-sn-glycero-3-phosphocholine (DPPC), and 1,2-dipalmitoyl-sn-glycero-3-phosphoethanolamine-N-[3-(2-pyridyldithio)propionate] (PDP-PE) were obtained from Avanti Polar Lipids (Alabaster, AL). Chloro(triethylphosphine)gold(I), Et₃PAuCl, was purchased from Gelest, Inc. or Sigma Aldrich and used as received. Gold nanoparticles (AuNPs, 5 nm) was obtained from nanoComposix (San Diego, CA). Purified monomeric human apolipoprotein A-I (apoA-I) was obtained from MyBioSource (San Diego, CA). Ultrapure deionized (DI) H₂O (18.2 MΩ·cm resistivity) was obtained from a Millipore system (Milli-Q Biocel). Auranofin was purchased from Sigma-Aldrich (Milwaukee, WI).

Vortexing was caried out on an analog vortex mixer (VWR, Radnor, PA). Sonication was carried out in a water-filled Branson 2510 Ultrasonic Sonicator (St. Louis, MO, USA). Centrifugation was carried out in an Eppendorf Centrifuge 5415 R (Eppendorf AG, Hamburg, Germany) equipped with an FA-45-24-11 rotor. All centrifugations were carried out at 25 at 8000-11000 rpm (6010-11363 g) for 10-30 minutes. Nanoparticle incubations at room temperature (rt) are carried out on a Corning LSE flat-bottom orbital shaker (Glendale, AZ) set at 60 rpm. Tangential flow filtration (TFF) was carried out on a KrosFlo Research Ili TFF System, Repligen model 900-1613 using a commercially available membrane (50 kDa MWCO, 11 cm² surface area; Spectrum Laboratories, Inc., Rancho Dominguez, CA).

Dynamic light scattering (DLS) and zeta potential measurements were carried out on a Zetasizer Nano ZS (Malvern Instruments, Malvern, UK) equipped with a He-Ne laser (633 nm). Non-invasive backscatter method (detection at 173° scattering angle) was used. Correlation data were fitted, using the method of cumulants, to the logarithm of the correlation function, yielding the diffusion coefficient, D. The hydrodynamic diameters (D_{H}) of nanoparticles were calculated using D and the Stokes-Einstein equation (D_{H}=k_{B}T/3πηD, where k_{B} is the Boltzmann constant, T is the absolute temperature, and η is the solvent viscosity (η=0.8872 cP for water)). The polydispersity index (PDI) of the nanoparticles-represented as 2c/b², where b and c are first- and second-order coefficients, respectively, in a polynomial of a semi-log correlation function—was calculated by cumulants analysis. Size distribution of the nanoparticles was obtained by non-negative least squares (NNLS) analysis.

TEM imaging was carried out with a Hitachi HD2300 Scanning Transmission Election Microscope (STEM). Flow cytometry was performed using a BD LSRFortessa cell analyzer.

UV-vis spectroscopic data were collected on an Agilent 9453 spectrometer. Mass spectrometric data were collected as negative and positive ions using an LC-TOF mass spectrometer (Agilent 6545 Q-TOF LC/MS). Data from Agilent HPLC and Agilent Q-TOF instruments were processed using Agilent MassHunter Workstation software version10.0.

### Materials and Protocols for In Vitro Studies.

Media and cell culture reagents. All Fisher-brand cell culture consumables (flasks, plates, and serological pipettes), media, buffers, serum, and dissociation reagents were purchased from Fisher Scientific (Waltman, MA).

Cell lines and conditions. ES-2 ovarian cancer cells and 786-O ccRCCa clear cell renal cell carcinoma cancer cells were purchased from American Type Culture Collection (ATCC, Manassas, VA, USA). Ramos (SR-B1-positive Burkitt's lymphoma cell line), SUDHL4 (SR-B1-positive germinal center diffuse large B cell lymphoma cell line) and Jurkat (SR-B1-negative T cell lymphoma cell line) cells were also purchased from ATCC (American Type Culture Collection, Manassas, VA). THP1-Dual cells were purchased from InvivoGen (San Diego, CA).

ES-2 cells were cultured using McCoy's 5A supplemented with 10% vol % fetal bovine serum (FBS), 4 mM L-glutamine, penicillin (100 unit/mL), and streptomycin (0.1 mg/mL). All other cells were cultured using RPMI 1640 supplemented with 10 vol % fetal bovine serum (FBS), 4 mM L-glutamine, 25 mM HEPES, penicillin (100 unit/mL), and streptomycin (0.1 mg/mL). THP1-Dual cells were maintained in 10 μg/mL Blasticidin and 100 μg/mL Zeocin to maintain selection. All cell-culture experiments were carried out using the aforementioned cell-specific media in an incubator operating at 37 °C and in a humidified atmosphere containing 5 vol % CO₂. 786-0 cells were harvested using a TrypLE Express (1×) solution and ES-2 cells were harvested using 1 mM EDTA solution containing 6.8 mM sodium chloride, 333 μM sodium bicarbonate, 278 μM D-glucose, and 268 μM potassium chloride.

In vitro cellular viability assay. ES-2 (2,500 cells/well), 786-0 ccRCCa (400 cells/well), Ramos (16,000 cells/well), SUDHL4 (16,000 cells/well), Jurkat (16,000 cells/well), CWR-R1 (1,000 cells/well), LNCaP (2,000 cells/well), and 22rv1 (2,000 cells/well) were plated into 96 well plates. Adherent ES-2, CWR-R1, LNCaP, 22rv1, and 786-0 cells were incubated for 24 h. Media in the wells was then replaced with pre-prepared media containing the appropriate drug formulations at the appropriate drug concentration. Ramos, SUDHL4, and Jurkat cells are suspension cell lines and were plated and treated on the same day. After 72 h, cell viability was measured via an MTS cell-proliferation assay (CellTiter 96 Aqueous One, Promega; Madison, WI) and the relative cell-survival percentages compared to the vehicle control were plotted against the concentration of either the AuNP or the oc-HDL NPs in logarithmic scale. The data reported represent an average of three measurements.

In vitro cellular inflammation assay. THP1-Dual cells (100,000 cells/well) were plated into 96 well plates and at the same time treated with media containing the appropriate drug formulations at the appropriate drug concentration with or without 1 ng/mL lipopolysaccharide (LPS; O55:B5) from Sigma-Aldrich (St. Louis, MO). After 24 h, 20 μL of cells and media were transferred to a new 96 well plate and 180 μL of QUANTI-Blue Media (InvivoGen; San Diego, CA) was added to each well. Plates were incubated at 37 °C in a humidified 5 vol % CO₂ incubator for 1 h. Secreted embryonic alkaline phosphatase (SEAP) levels as a readout of NFκB activity were determined by reading the absorbance at 655 nm in a Biotek Synergy 2 plate reader (Winooski, VT) and the relative NFκB percentages compared to [LPS+vehicle] control were plotted against the AuNP concentration in logarithmic scale. The data reported represent an average of three measurements.

### Example 1: Synthesis of Compound 1 (thiolate cholesterol)gold(triethylphosphine) (cholS-AuPEt₃)

Compound 1 was synthesized as follows. A 1-dram vial equipped with a magnetic stir bar was charged with Et₃PAuCl (0.15 g, 0.43 mmol, 1 equiv), thiocholesterol (0.15 g, 0.43 mmol, 1 equiv), and CH₂Cl₂ (1 mL). The reaction mixture was cooled down to 0 °C on ice; a solution of K₂CO₃ (74 mg, 0.51 mmol, 1.2 equiv) in H₂O (1 mL to make a 0.51 M aqueous solution) was added; and the resulting biphasic mixture was gradually warmed up to the room temperature and stirred for an additional 2 h. The organic layer was carefully separated, and the aqueous layer was further extracted with CH₂Cl₂ (4×2 mL). The combined organics were dried over Na₂SO₄, filtered, and concentrated in vacuo to afford the desired product, which was used without further purification.

ESI-TOF HRMS m/z 717.3896, [(M+H+); calcd for C33H61AuPS: 717.3897].

### Example 2: Synthesis of Compound 2 (DPPTE-AuPEtA)

Following the synthetic procedure for Compound 1, the Au-lipid complex Compound 2 was prepared from 16:0 Ptd thioethanol (DPPTE, 14.6 mg, 20 μmol, 1 equiv), Et₃PAuCl (7.0 mg, 20 μmol, 1 equiv), K₂CO₃ (3.3 mg, 24 μmol, 1.2 equiv) solution in H₂O (1 mL to make a 24 mM aqueous solution) and CH₂Cl₂ (1.6 mL). The desired compound was obtained and used without further purification.

ESI-TOF HRMS (ESI-TOF) m/z 1021.5243 ([M−Na]⁺); calcd for C₄₃H₈₆AuO₈P₂S: 1021.5184].

### Example 3: Dissolved HDL NP Synthesis

Rationale. Synthetic high-density lipoprotein-like nanoparticles (HDL NP) were synthesized using a AuNP as a core scaffold to support the self-assembly of phospholipids and apoA-I. One of the phospholipids (PLs) on the surface of the AuNP scaffold, 16:0 PDP-PE, has a disulfide-containing headgroup, so that it will chemisorb in a “head-down” fashion to the AuNP surface to form PLS-AuNP moieties. (The PLS notation denotes the phospholipid (PL) thiolate (S) moiety that complexes to a Au^{I} moiety on the AuNP surface after cleavage of the disulfide bond in the 16:0 PDP-PE lipid.) A second type of PL (e.g., DPPC) can then be oriented in a “tail-to-tail” arrangement vs the “head-down” PLS moiety, with its zwitterionic choline headgroup being directed outward toward the aqueous solvent. Addition of apoA-I to the assembly mixture additionally favors the self-assembly of this second type of lipid with the AuNP-surface-immobilized PL layers, leading to the formation of synthetic Au⁰-HDL NPs. The apoA-I can exist on the surface of these synthetic Au⁰-HDL NPs as dimers, trimers, or even higher order oligomers.

Potassium cyanide (KCN) is known to rapidly oxidize Au⁰ (4 Au+8 KCN+O₂+2 H₂O→4 K[Au(CN)₂]+4 KOH). Accordingly, it was hypothesized that addition of KCN to the synthetic Au⁰-HDL NPs would result in the dissolution of the core AuNP, leaving the PLS-Au complexes in its core, surrounded by the second type of PL and apoA-I. Further, because gold salt has been shown to have anti-cancer and anti-inflammatory properties, the efficacy of the parent Au⁰-HDL NPs in a cancer-cell-killing assay was compared against that of the HDL NPs where KCN has been added to dissolve the AuNP core.

In a typical synthesis, 5 nm gold NPs (35 mL of a 70 nM Au solution) were mixed with a 5-fold molar excess apoA-I in a 50 mL conical tube. AuNP concentration was determined using Beer's law and UV-vis spectroscopy, where the 5 nm AuNPs have a characteristic absorption at at λₘₐₓ=520 nm (ε=9.696×10⁶ M⁻¹ cm⁻¹). The resulting mixture was incubated for 1 h at room temperature (rt) on a flat-bottom shaker. Next, 20% ethanol (7 mL) was added and the whole mixture is vortexed for 5 s. A solution of PDP-PE in ethanol (612.5 μL of a 1 mM solution) was then added to this mixture to achieve a final lipid/AuNP molar ratio of 250. The resulting mixture was vortexed (5 s), followed by the addition of a solution of DPPC in ethanol (612.5 μL of a 1 mM solution) to again achieve a final DPPC/AuNP molar ratio of 250. The resulting HDL NP mixture was vortexed (5 s) and incubated overnight at rt on a flat-bottom shaker. In the morning, the HDL NPs were isolated and concentrated via TFF, and stored at 4 °C until use. The concentration of the HDL NPs were measured using UV-vis spectroscopy, where the 5 nm AuNPs have a characteristic absorption at λₘₐₓ=520 nm (ε=9.696×10⁶ M⁻¹ cm⁻¹).

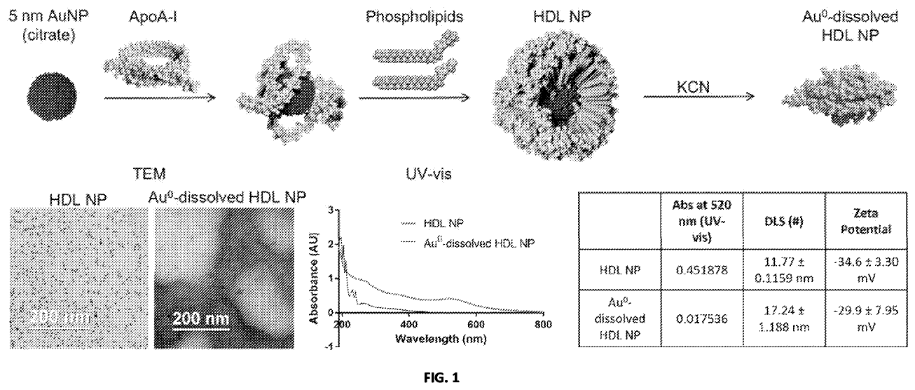

As discussed above, KCN can be added to the solution of Au⁰-HDL NPs to dissolve the AuNP core. In a typical preparation, Au⁰-HDL NP (1 mL of a 1 μM Au⁰ solution) was combined with an aliquot of aqueous KCN (20 μL of a 1 M solution) and incubated overnight at rt on a flat-bottom shaker. The AuNP core in the Au⁰-HDL NP was shown to have been dissolved by visual inspection (the solution changes color from red to yellow), and by UV-vis spectroscopy whereby the absorption at λₘₐₓ=520 nm disappears. Presumably, due to the scaffolding properties of the surface apoA-Is and PLs, the resultant dissolved HDL NPs can increase slightly in size, as measured by TEM and DLS (FIG. 1), and retain the negative surface charge via zeta potential measurements (FIG. 1).

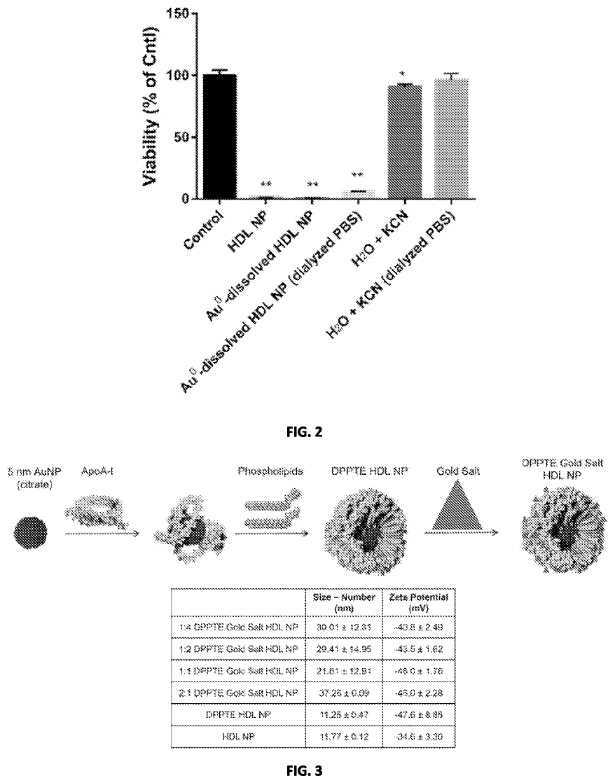

The-HDL NP and Au⁰-dissolved HDL NPs (i.e., [Au⁰-HDL NP treated with KCN] and [Au⁰-HDL NP treated with KCN and dialyzed with PBS]) were tested against clear cell renal cell carcinoma cells (ccRCCa) as described in the Materials and protocols for in vitro studies section above. Data show potent killing of these cells with all three formulations while the controls (PBS-dialyzed and non-dialyzed aqueous KCN) did not exhibit a profound decrease in cell viability (FIG. 2).

### Example 4: Synthesis of Au-Salt-Containing HDL NPs from DPPTE

Rationale. Instead of forming HDL NPs containing PLS-gold salts in situ by dissolving the AuNP core of Au⁰-HDL NP, an approach to synthesize Au-salt-containing HDL NPs by carrying out the initial Au0-HDL NP synthesis was also developed, using a single PL containing a thiol (—SH) headgroup, such as DPPTE. Accordingly, the HDL NP would now have “head-down” PLs due to the reaction of the —SH headgroup with the AuNP to make PLS-Au bond; and, by virtue of bilayer-lipid assembly, would also have a second, outer layer comprising DPPTE displaying its —SH headgroup toward the aqueous solution. Addition of Et₃PAuCl would result in the formation of a layer of PLS-AuPEt₃ salts on the surface of the HDL NP. TFF or dialysis can be used to purify the new Au-salt-containing HDL NP from the unreacted gold salt. In this second synthesis, the number of Au-based drugs per HDL NP was expected to be higher than that in the Au⁰-dissolved HDL NP synthesis.

In a typical synthesis, 5 nm AuNPs (35 mL of a 70 nM Au solution) were mixed with a 5-fold molar excess of apoA-I in a 50 mL conical tube. AuNP concentration was determined using Beer's law and UV-vis spectroscopy, where the 5 nm AuNPs have a characteristic absorption at λmax=520 nm (ε=9.696×106 M−1 cm−1). The resulting mixture was incubated for 1 h at rt on a flat-bottom shaker. Next, 20% ethanol (7 mL) was added and the whole mixture was vortexed for 5 s. A solution of DPPTE in ethanol (875 μL of a 1 mM solution) was then added to this mixture to achieve a final lipid/AuNP molar ratio of 500. The resulting DPPTE HDL NPs mixture was briefly vortexed (5 s), incubated overnight at rt on a flat-bottom shaker, and then purified and concentrated via TFF. Next, the crude DPPTE HDL NPs (250 nM) were combined with ethanol solutions of Et₃PAuCl of various concentrations (9.1, 18.3, 36.6, and 73.1 μM) and aqueous K₂CO₃ solution (6 μM) and incubated overnight at rt on a flat-bottom shaker. In the morning, the mixture (~1 mL) was then dialyzed with 100 kDa dialysis devices in PBS (15 mL) for 24 h at rt on a flat-bottom shaker, with one change of fresh PBS at the 6 h point. The purified DPPTE-Au-containing HDL NPs were stored at 4 °C.

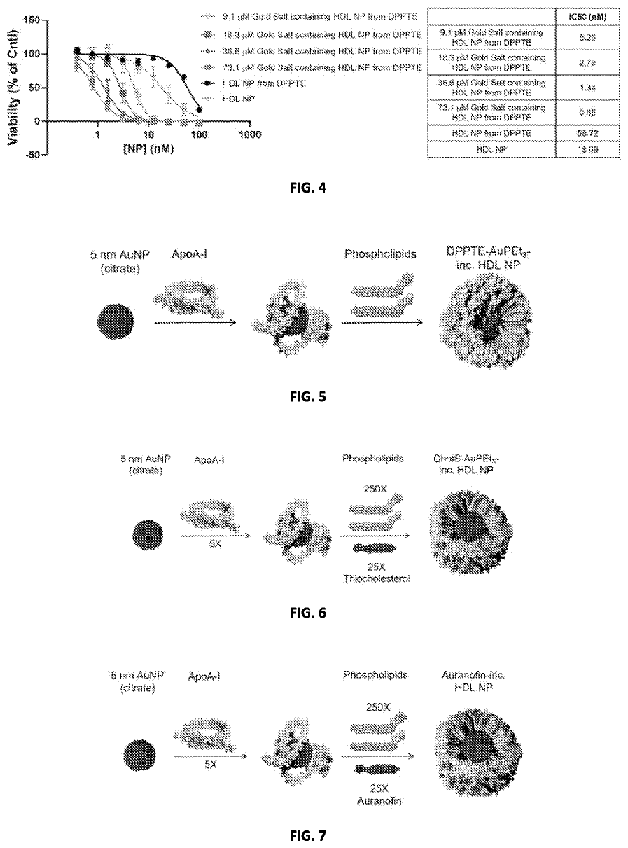

DLS data showed that the DPPTE-Au-containing HDL NPs are larger in size versus DPPTE HDL NPs and the parent Au⁰-HDL NPs made with 16:0 PDP-PE and DPPC, which is consistent with them having the much larger Et₃PAu-containing headgroup on the solvent-facing side of the surface lipid bilayer. In addition, the surface charges for the two former DPPTE-containing compositions were more negative than that for the latter, which can be explained by the presence of the negatively charged DPPTE and DPPTE-AuPEt₃ moieties, respectively, versus the zwitterionic DPPC. As Et₃PAuCl was added at different ratios to the solvent-facing DPPTE layer to form DPPTE-AuPEt₃ moieties (Et₃PAuCl/DPPTE ratio was reduced from 2:1 to 1:4), the surface charge of the resulting DPPTE-Au-containing HDL NPs becomes less negative compared to that for the parent DPPTE HDL NPs with no added Et₃PAuCl (FIG. 3). Regarding their abilities to kill ccRCCa cells (determined as described in the Materials and protocols for in vitro studies section above), the DPPTE-Au-containing particles had very low IC₅₀ values (0.85-5.25 nM) versus those for the DPPTE HDL NPs (58.72 nM) or the Au⁰-HDL NP made with DPPC (18.09 nM) (FIG. 4). These data are consistent with the former having better cytotoxicity from the presence of the DPPTE-AuPEt₃ gold salt.

### Example 5: Synthesis of Au⁰-HDL NPs that Directly Incorporate LS-AuPEt₃ Gold Salts

Rationale. The HDL NP platform is powerful for binding cholesterol, PLs, and targeting scavenger receptor type B-1 (SR-B1). The SR-B1 cell-surface receptor is highly expressed on cancer and immune cells, for instance. As a result, the HDL NP platform is ideal for incorporating drugs, especially those that have similar structures to cholesterol and PLs, and delivering these drugs to immune and cancer cells. We sought to directly incorporate both thiolated PL- and cholesterol-gold salt molecules (DPPTE-AuPEt₃ and cholS-AuPEt₃, respectively) as molecular building blocks of HDL NPs. The resulting LS-AuPEt₃-incorporated HDL NPs were ideally suited for targeted delivery of the LS-AuPEt₃ cargo to SR-B1-positive target cells.

Synthesis of Au0-HDL NPs that Directly Incorporate DPPTE-AuPEt3 Gold Salt Phospholipid.

In a typical synthesis, 5 nm AuNPs (17 mL of a 70 nM Au solution) were mixed with a 5-fold molar excess of apoA-I in a 20 mL glass vial. AuNP concentration was determined using Beer's law and UV-vis spectroscopy, where the 5 nm AuNPs had a characteristic absorption at at λₘₐₓ=520 nm (ε=9.696×10⁶ M⁻¹ cm⁻¹). The resulting mixture was incubated for 1 h at rt on a flat-bottom shaker at 60 rpm. An aliquot of a CH₂Cl₂ solution of PDP-PE (297.5 μL of a 1 mM solution) was then added to this mixture to achieve a final lipid/AuNP molar ratio of 250. The resulting mixture was vortexed (15 s), followed by the addition of a solution of DPPTE-AuPEt₃ in CH₂Cl₂ (297.5 μL of a 1 mM solution) to again achieve a final DPPTE-AuPEt₃/AuNP molar ratio of 250. The resulting HDL NP mixture was vortexed (30 s) and sonicated (30 s) ten times to produce an emulsion. Once emulsified, the dichloromethane was boiled off in a water bath set at 42 °C with stirring. The resulting DPPTE-AuPEt₃-incorporated HDL NPs were incubated overnight at rt on a flat-bottom shaker at 60 rpm. In the morning, the HDL NPs were isolated and concentrated via TFF, and stored at 4 °C until use. The concentration of these DPPTE-AuPEt₃-incorporated HDL NPs was measured using UV-vis spectroscopy, where the AuNPs have a characteristic absorption at λₘₐₓ=520 nm (ε=9.696×10⁶ M⁻¹ cm⁻¹).

Synthesis of Au⁰-HDL NPs that Directly Incorporate Thiocholesterol-AuPEt₃ Gold Salt.

In a typical synthesis, 5 nm AuNPs (17 mL of a 70 nM Au solution) were mixed with a 5-fold molar excess of apoA-I in a 20 mL glass vial. AuNP concentration was determined using Beer's law and UV-vis spectroscopy, where the 5 nm AuNPs had a characteristic absorption at at λₘₐₓ=520 nm (ε=9.696×10⁶ M⁻¹ cm⁻¹). The resulting mixture was incubated for 1 h at rt on a flat-bottom shaker at 60 rpm. An aliquot of a CH₂Cl₂ solution of PDP-PE (297.5 μL of a 1 mM solution) was then added to this mixture to achieve a final lipid/AuNP molar ratio of 250. The resulting mixture was vortexed (15 s), followed by the addition of a solution of DPPC in CH₂Cl₂ (297.5 μL of a 1 mM solution) to again achieve a final DPPC/AuNP molar ratio of 250. The resulting mixture was again vortexed (15 s), followed by addition of a solution of cholS-AuPEt₃ in CH₂Cl₂ (29.75 μL of a 1 mM solution) to achieve a final cholS-AuPEt₃/Au⁰ molar ratio of 25. Further vortexing (30 s) and sonication (30 s) then produced an emulsion. Next, the dichloromethane was boiled off in a water bath at 42 °C with stirring. The resulting cholS-AuPEt₃-incorporated HDL NPs were incubated overnight at rt on a flat-bottom shaker at 60 rpm. In the morning, the HDL NPs were isolated and concentrated via TFF and stored at 4 °C until use. The concentration of these cholS-AuPEt₃-incorporated HDL NPs was measured using UV-vis spectroscopy, where the AuNPs have a characteristic absorption at λₘₐₓ=520 nm (ε=9.696×10⁶ M⁻¹ cm⁻¹).

Synthesis of Au⁰-HDL NPs that Directly Incorporate Auranofin Gold Salt.

In a typical synthesis, 5 nm AuNPs (35 mL of a 70 nM Au solution) were mixed with 5-fold molar excess of apoA-I in a 50 mL conical tube. AuNP concentration was determined using Beer's law and UV-vis spectroscopy, where the 5 nm AuNPs had a characteristic absorption at at λₘₐₓ=520 nm (ε=9.696×10⁶ M⁻¹ cm⁻¹). The resulting mixture was incubated for 1 h at rt on a flat-bottom shaker. Next, 20% ethanol (7 mL) was added and the whole mixture is vortexed for 5 s. A solution of PDP-PE in ethanol (437.5 μL of a 1 mM solution) was then added to this mixture to achieve a final lipid/AuNP molar ratio of 250. The resulting mixture was vortexed (5 s), followed by the addition of a solution of DPPC in ethanol (437.5 μL of a 1 mM solution) to again achieve a final DPPC/AuNP molar ratio of 250. The resulting mixture is vortexed (5 s), followed by the addition of a solution of auranofin in ethanol (43.75 μL of a 1 mM solution) to achieve a final auranofin/AuNP molar ratio of 25. The resulting HDL NP mixture was vortexed (5 s) and incubated overnight at rt on a flat-bottom shaker. In the morning, the HDL NPs were isolated and concentrated via TFF, and stored at 4 °C until use. The concentration of the HDL NPs was measured using UV-vis spectroscopy, where the 5 nm AuNPs have a characteristic absorption at λₘₐₓ=520 nm (ε=9.696×10⁶ M−1 cm⁻¹).

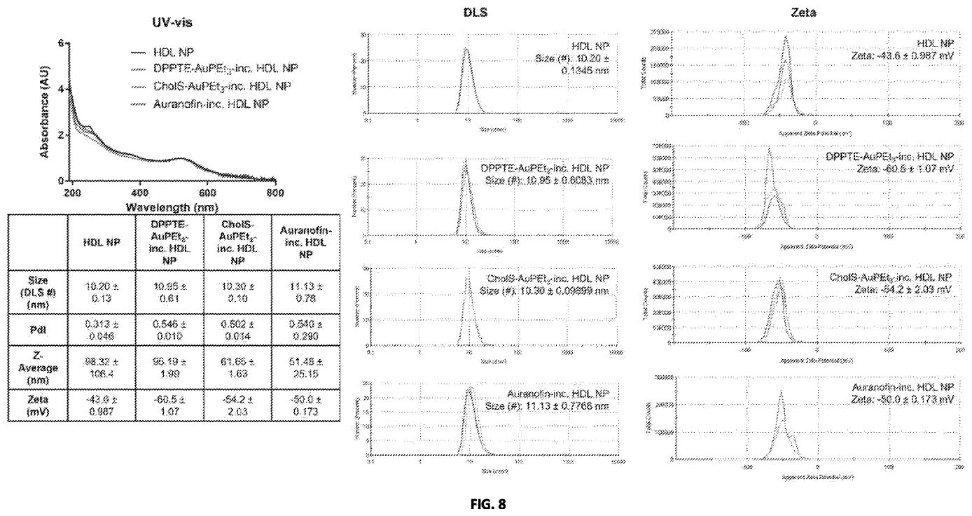

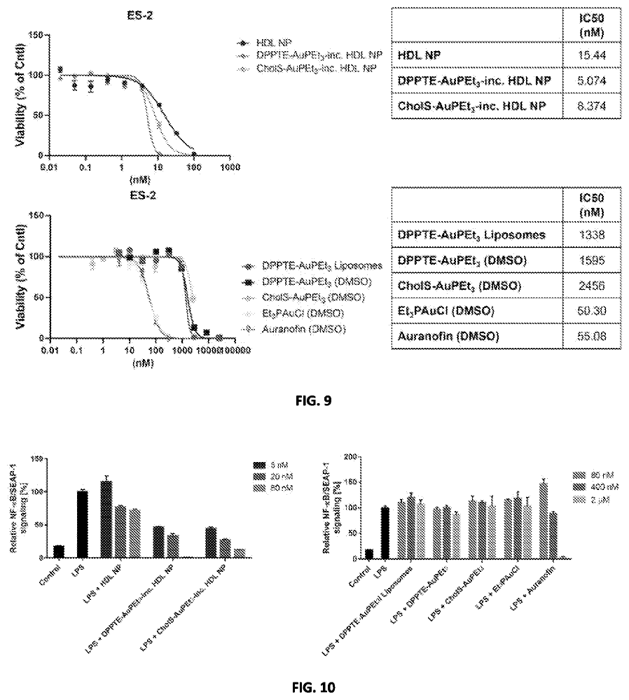

The synthetic schemes for each of the aforementioned LS-AuPEt₃-incorporated HDL NPs are shown in FIGS. 5, 6, and 7. Data showing the size and charge of each of the new materials is shown in FIG. 8. Each of these materials was then tested regarding their ability to kill ES-2 ovarian cancer cells (determined as described in the Materials and protocols for in vitro studies section above). Data (FIG. 9) showed that they are quite potent at killing ES-2 cells. Notably, the LS-AuPEt₃-incorporated HDL NPs were superior to either the addition of the free LS-AuPEt₃ molecules or the LS-AuPEt₃ molecule delivered in liposomal form.

Also, gold salt compounds are known to target cellular redox enzymes (e.g., TXNRD1) and also to inhibit the cell inflammatory response by reducing NF-κB transcription and the production of inflammatory cytokines. Using an in vitro human macrophage cell model, each of the novel compounds was tested to assess reduction in activation of NF-κB in the presence of a powerful stimulator of NF-κB activity called lipopolysaccharide (LPS) (determined as described in the Materials and protocols for in vitro studies section above). Data (FIG. 10) showed that LPS greatly increased NF-κB activity and several of the HDL NP formulations reported herein were effective at reducing NF-κB activity. The most significant reduction was observed with the HDL NPs that directly incorporated the DPPTE-AuPEt₃ or cholS-AuPEt₃ complexes. At low nM concentrations, these two materials suppressed NF-κB activity to the level measured for the control or below. Not surprisingly, they were also superior to the other liposomal formulation, free drug, or auranofin.

### Example 6: Synthesis of Oc-HDL NPs that Directly Incorporate LS-AuPEt3 Gold Salts

### Rationale

Similar to the HDL NP materials, the oc-HDL NPs are powerful from the standpoint of specific targeting SR-B1 and that they are capable of binding and loading cholesterol and PLs. Thus, directly incorporating into them drugs that are similar in structure to these molecules, such as DPPTE-AuPEt₃ and cholS-AuPEt₃, will create ideal vehicles that can deliver these drugs to immune and cancer cells.

Synthesis of DPPTE-AuPEt3-Incorporated and chols-AuPEt3-Incorporated Oc-HDL NP.

Prior to each synthesis of DPPTE-AuPEt₃-inc oc-HDL NPs, fresh solutions of each reagent were prepared. Aqueous solutions of apoA-I (1.74 mg/mL) and sodium cholate (0.5 M), and dichloromethane (DCM) solutions of the organic core scaffold PE-S₄ (0.135 mg/mL), and the phospholipids DOPC (5 mM) and DPPTE-AuPEt₃ (1 mM) were prepared in sufficient volumes for the required syntheses.

For a 1 nmol scale synthesis, the following reagents were added to a 6 mL glass vial: 8.93 μL of PE-S₄ (0.135 mg/mL), 32.2 μL of apoA-I (1.74 mg/mL), 29.4 μL of sodium cholate (0.5 M), 505.2 μL of water, and 180 μL of phospholipids/DCM. The DOPC/DPPTE-AuPEt₃ ratio was adjusted to accommodate the following ratios while maintaining a strict 300 to 1 ratio of total phospholipids to PE-S4 core: 300/0 (60 μL DOPC, 0 μL DPPTE-AuPEt₃, 120 μL DCM); 270/30 (54 μL, 30 μL, 96 μL); 250/50 (50 μL, 60 μL, 70 μL); 225/75 (45 μL, 75 μL, 60 μL); 200/100 (40 μL, 100 μL, 40 μL), and 150/150 (30 μL, 150 μL, 0 μL). The final mixture in each glass vial was 3/1 water/dichloromethane. Each vial was then subjected to 3 rounds of vortexing (20 s) followed by sonication (20 s). Afterwards, a magnetic stir bar was added to each vial and the vials were stirred on a magnetic stir plate overnight with the caps off to allow the dichloromethane fraction to evaporate. Following overnight incubation, the nanoparticle solutions were transferred to Eppendorf tubes and centrifuged at 10,000 rpm for 10 minutes at room temperature. The supernatant was transferred to dialysis tubes with a 50 kDa MWCO (Tube-O-DIALYZER, cat no. 786-624; G-Biosciences, St. Louis, MO) and dialyzed against 1×PBS for 24 h. The dialysis buffer was replaced with fresh 1×PBS approximately 8-12 h into the dialysis. The concentration of apoA-I in the solution was measured using the BCA protein assay, as the added apoA-I was the only protein present in the oc-HDL NPs. The nanoparticle concentration was the estimated by converting the g/L concentration of apoA-I to molarity and dividing by 2.23, the previously determined number of apoA-I molecules per nanoparticle for the synthesis method and organic core scaffold used above.

For the synthesis of cholS-AuPEt₃-incorporated oc-HDL NPs, as with the DPPTE-AuPEt₃-inc oc-HDL NP synthesis, all reagents (DOPC and PE-S₄ in DCM, apoA-I and sodium cholate in water) were prepared fresh. In addition, the CholS-AuPEt₃ was dissolved in DCM at a concentration of 1 mM. For a 1 nmol scale synthesis, the following reagents were added to a 6 mL glass vial: 8.93 μL of PE-S₄ (0.135 mg/mL), 32.2 μL of apoA-I (1.74 mg/mL), 18.5 μL of sodium cholate (0.5 M), 60 μL of DOPC (5 mM), 306.1 μL of water, and 50 μL of cholS-AuPEt₃ in DCM. Two different ratios of cholS-AuPEt₃/PE-S₄ were used: 50/1 (50 μL of 1 mM cholS-AuPEt₃, 0 μL of DCM) and 25/1 (25 μL, 25 μL). Following addition of all reagents into the glass vials, the nanoparticles were subjected to the same synthesis and purification process as the DPPTE-AuPEt₃-inc oc-HDL NPs, described above.

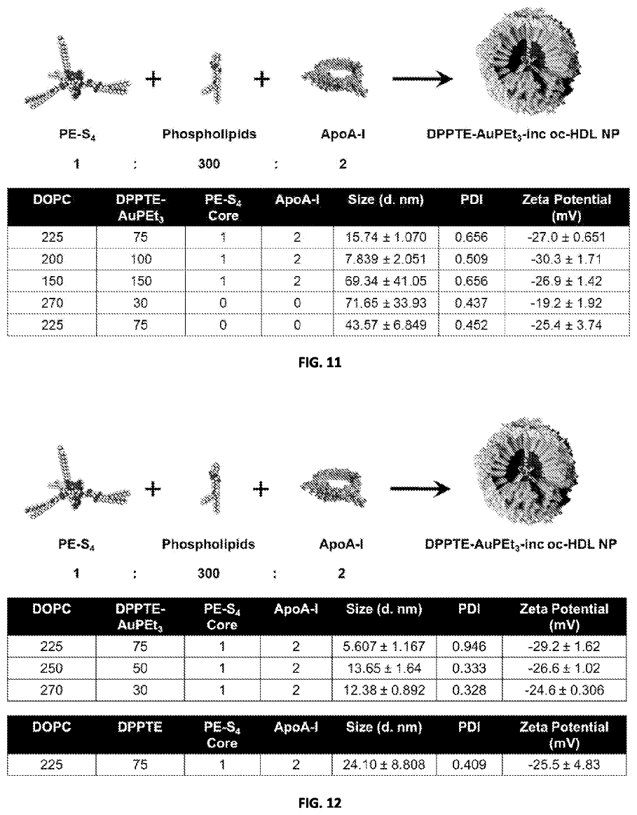

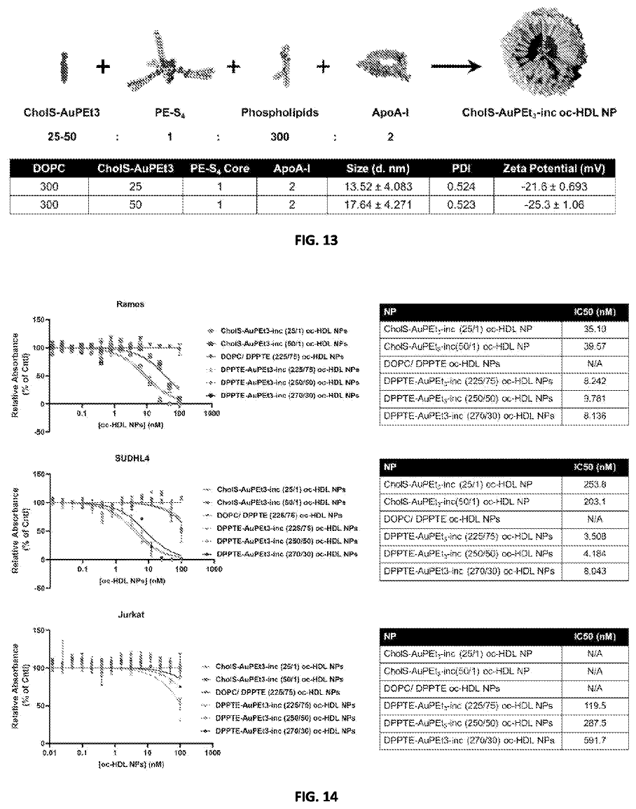

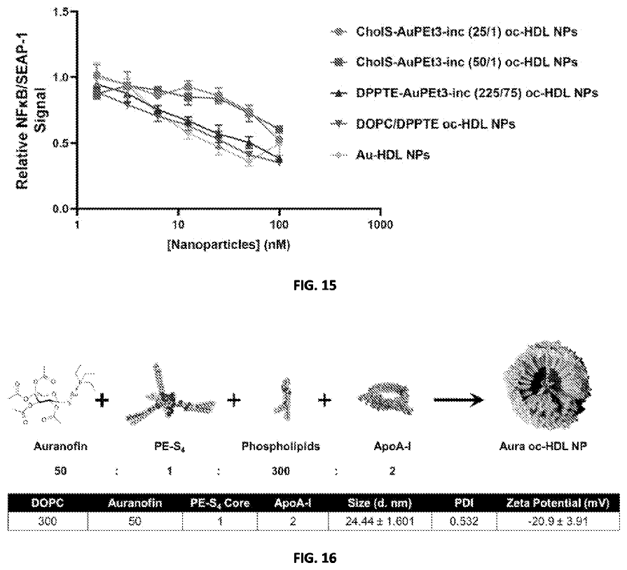

FIGS. 11-13 show the synthetic scheme of the DPPTE-AuPEt₃-inc oc-HDL NPs and cholS-AuPEt₃-inc oc-HDLNPs, and data demonstrating their characterization and functionality. Most impressively, DPPTE-AuPEt₃-inc oc-HDL NPs showed low nM IC₅₀ values in their ability to induce cell death in SR-B1-positive lymphoma cell lines (Ramos, SUDHL4) while demonstrating significantly less effectiveness against the SR-B1-negative Jurkat cell line. In contrast, control oc-HDL NPs, synthesized with unmodified DPPTE substituted in place of the DPPTE-AuPEt₃ phospholipid and without the LS-AuPEt₃, showed no cytotoxicity.

### Synthesis of Auranofin-Incorporated Oc-HDL NPs (Aura-Inc Oc-HDL NPs).

Prior to each synthesis of aura-inc oc-HDL NPs, fresh solutions of each reagent were prepared. Aqueous solutions of apoA-I (1.74 mg/mL) and sodium cholate (0.5 M); and dichloromethane (DCM) solutions of the organic core scaffold PE-S4 (0.135 mg/mL), the phospholipid DOPC (5 mM) and auranofin (1 and 2 mM) were prepared in sufficient volumes for the required syntheses in glass vials.

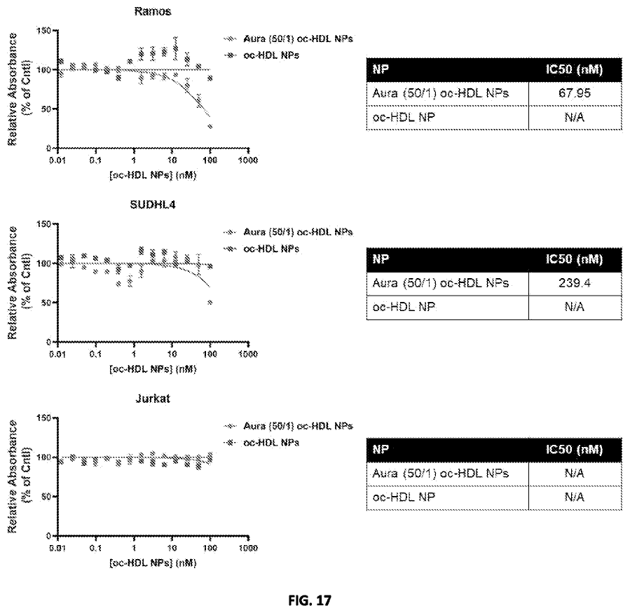

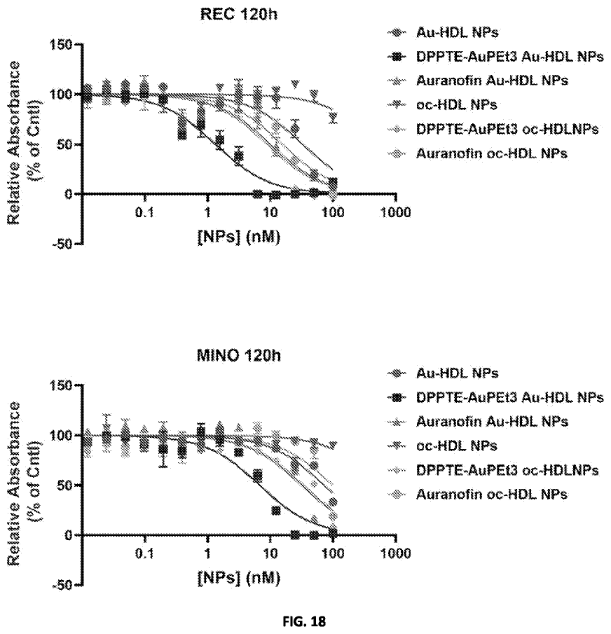

For a 1 nmol scale synthesis, the following reagents were added to a 6 mL glass vial: 8.93 μL PE-S₄ (0.135 mg/mL), 32.2 μL apoA-I (1.74 mg/mL), 15.6 μL sodium cholate (0.5 M), 252.6 μL water, 60 μL DOPC (5 mM) and 25 μL auranofin (1 mM or 2 mM). Two different ratios of auranofin/PE-S₄ were used: 50/1 (25 μL of 2 mM auranofin) and 25/1 (25 μL of 1 mM auranofin). Following the addition of all reagents into the glass vials, the nanoparticles were subjected to the same synthesis and purification process as the DPPTE-AuPEt₃-inc oc-HDL NPs, described above. FIG. 16-17 show the synthesis scheme of the aura-inc oc-HDL NPs and data demonstrating their characterization and functionality.

The auranofin delivered via the aura-inc oc-HDL NPs induced cell death in SR-B1-positive cell lines (Ramos, SUDHL4) while no change in viability was seen in the SR-B1-negative cell line (Jurkat). The IC₅₀ values for the aura-inc oc-HDL NPs were similar to those for the cholS-AuPEt₃-inc oc-HDLNPs.

### Example 7: Au-HDL Nanoparticles Induce Cell Death in Lymphoma Models

The HDL-NPs disclosed herein were found to be effective in inducing cell death in several in vitro lymphoma models. Cells from two Mantle Cell Lymphoma (MCL) cell lines (REC and MINO) were treated with gold-bearing HDL-NPs as disclosed herein in an assay described in the Materials and protocols for in vitro studies section above.

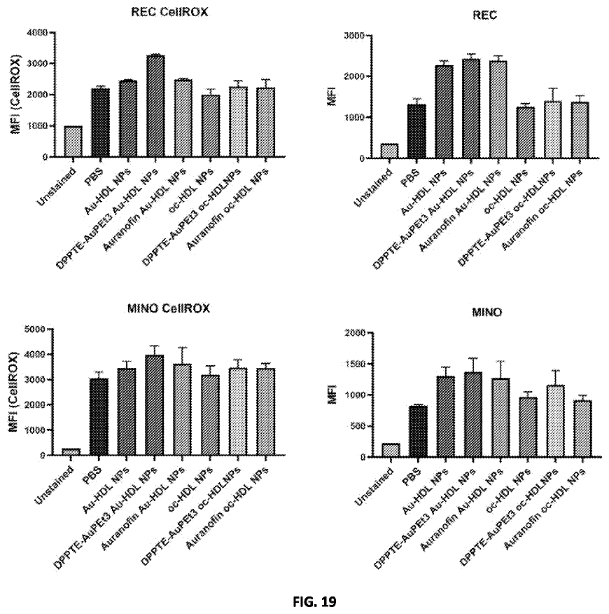

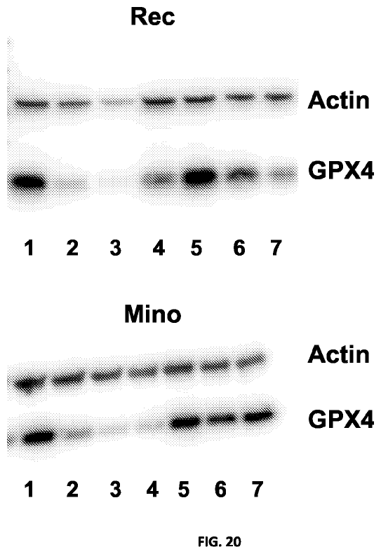

Both REC and MINO cells were found to be sensitive to HDL NP-induced cell death, and addition of DPPTE-AuPEt3 or Auranofin increased sensitivity (FIG. 18). Overall oxidative stress (ROX) was increased with DPPTE-AuPEt3 addition, although no change in lipid peroxide accumulation was noted between Au-HDL NPs and DPPTE-AuPEt3 Au-HDL NPs (FIG. 19).

The HDL-NPs disclosed herein were also found to reduce glutathione peroxidase 4 (GPX4) in the MCL cell lines tested above (FIG. 20). The HDL NPs templated using a gold core (AuNP) reduce GPX4 expression, as previously demonstrated in B-cell lymphoma and ovarian cancer cell lines (J. Rink et al., J. Biol. Chem. 296(C):100100 (2021). Y. Wang et al., Adv. Sci. (Weinh). 11(13):e2305212 (2024).). Incorporation of DPPTE-AuPEt3 or auranofin into AuNP templated HDL NPs did not alter the ability of the HDL NPs to reduce GPX4 expression. Organic core templated HDL NPs do not reduce GPX4 expression and incorporation of either DPPTE-AuPEt3 or auranofin did not lead to downregulation of GPX4.

### Example 8: Au-HDL Nanoparticles Induce Cell Death in Clear Cell Ovarian Cancer Models

Addition of Au-Salts to HDL NPs Decrease Cell Viability More than Addition of Au-Salts to Oc-HDL NPs in Clear Cell Ovarian Cancer Cells (ES2)

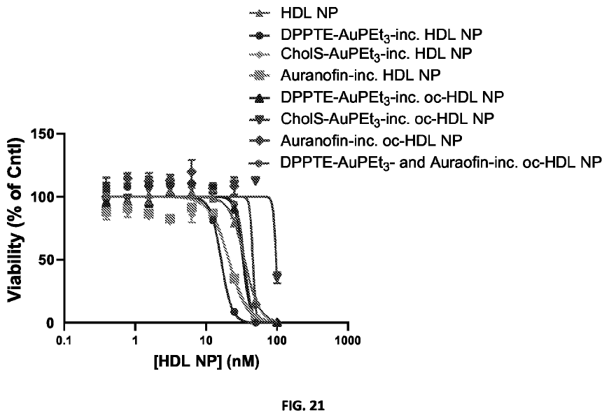

Addition of gold salts to HDL-NPs disclosed herein decreased cell viability more than the addition of gold salts to oc-HDL NPs disclosed herein in a clear cell ovarian cancer model (ES2 cell line). Cells were treated with gold-bearing HDL-NPs as disclosed herein in an assay described in the Materials and protocols for in vitro studies section above. Data are shown in FIG. 21, and IC50 data are presented in Table B:

TABLE B

| NP tested | IC50 (nM) |
|---|---|
| HDL NP | 34.94 |
| DPPTE-AuPEt₃-inc. HDL NP | 16.29 |
| CholS-AuPEt₃-inc. HDL NP | 20.99 |
| Auranofin HDL NP | 20.84 |
| DPPTE-AuPEt₃-inc. ocHDL NP | 33.01 |
| CholS-AuPEt₃-inc. ocHDL NP | ~97.19 |
| Auranofin ocHDL NP | ~45.76 |
| DPPTE-AuPEt₃/Auranofin-inc. ocHDL NP | 34.60 |

ES2 Cells Treated with Au-Salts and Ferrostatin-1 or DFO to Rescue Cell Death

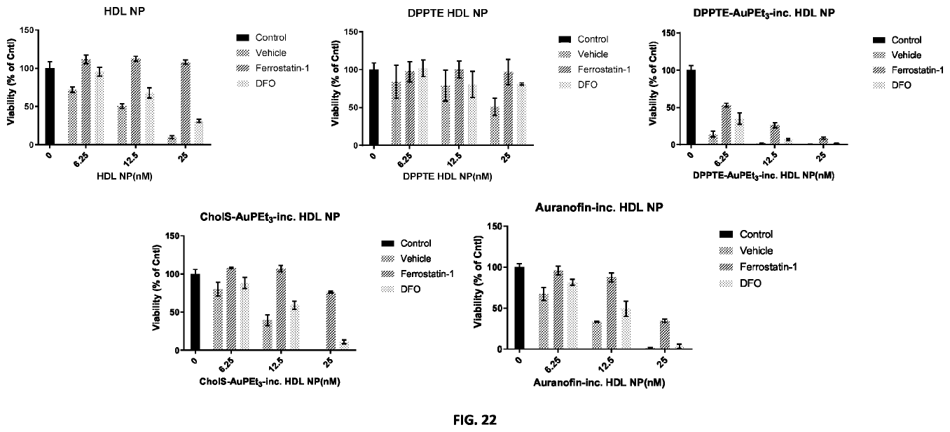

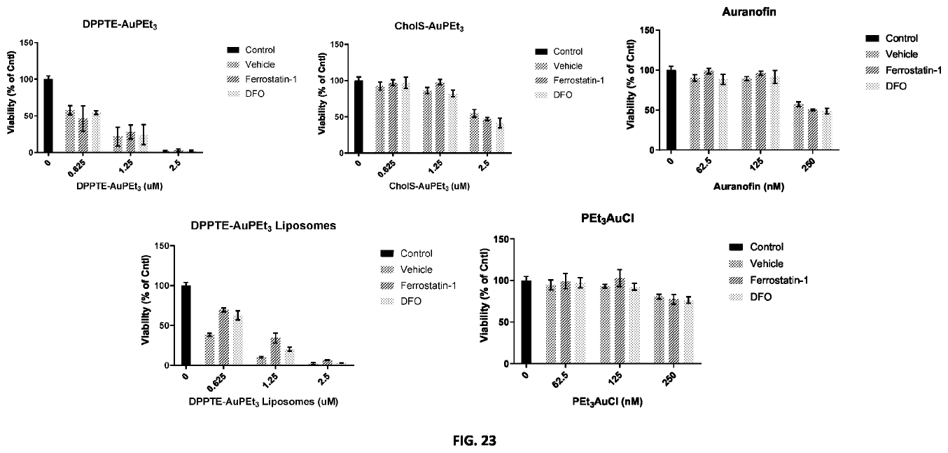

Au-salt containing HDL NP-induced cell death is rescued by addition of ferrostatin-1 or DFO, demonstrating a mechanism consistent with ferroptosis. Au-salt alone induced cell death is not rescued by ferrostatin-1 or DFO. Au-salts do not induce ferroptotic cell death. Cell viability in ES2 cells treated with either gold HDL-NPs or gold salts as disclosed herein was measured using an assay described in the Materials and protocols for in vitro studies section above, and data are presented in FIGS. 22 (gold HDL-NPs) and 23 (gold salts alone).

Addition of Au-Salts to HDL NPs Decrease Cell Viability More than Addition of Au-Salts to Oc-HDL NPs in Clear Cell Ovarian Cancer Cells (TOV-21G)

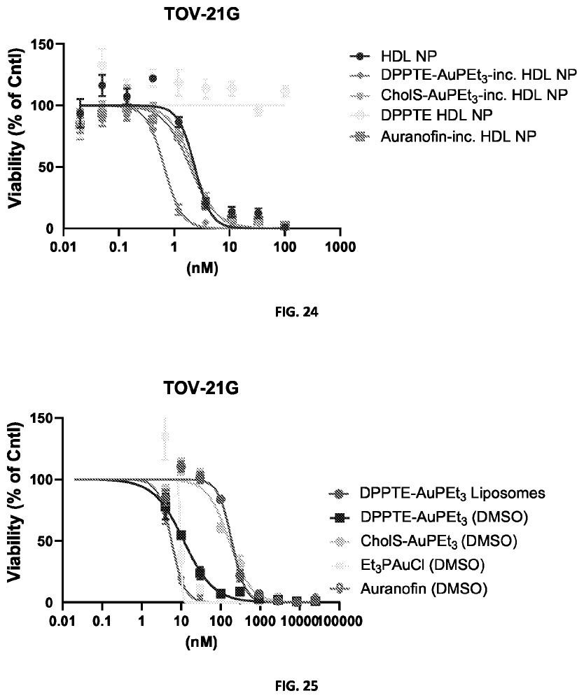

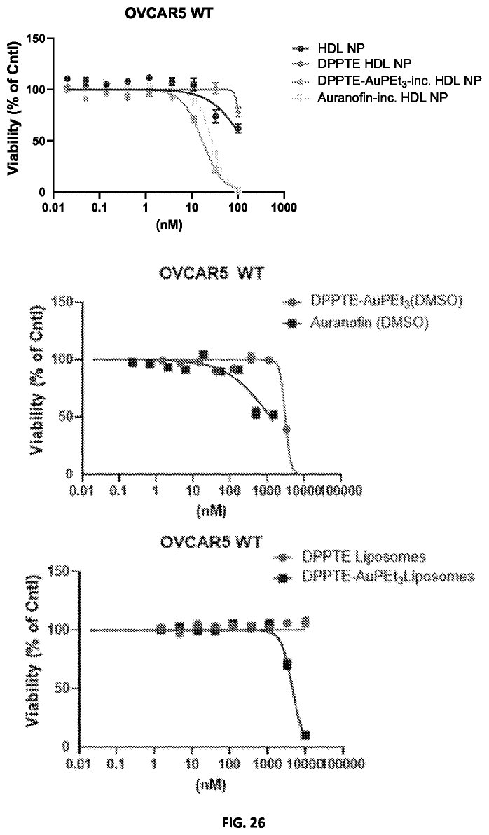

Addition of gold salts to HDL-NPs disclosed herein decreased cell viability more than the addition of gold salts to oc-HDL NPs disclosed herein in a clear cell ovarian cancer model (TOV-21Gcell line). Cells were treated with gold-bearing HDL-NPs or gold salts as disclosed herein in an assay described in the Materials and protocols for in vitro studies section above. Data are shown in FIGS. 24 (gold HDL-NPs) and 25 (gold salts alone), and IC50 data are presented in Table C:

TABLE C

| NP or Salt tested | IC50 (nM) |
|---|---|
| HDL NP | 2.361 |
| DPPTE-AuPEt₃-inc. HDL NP | 0.6617 |
| CholS-AuPEt₃-inc. HDL NP | 2.205 |
| DPPTE HDL NP | N/A |
| Auranofin-inc. HDL NP | 1.944 |
| DPPTE-AuPEt₃ Liposomes | 190.3 |
| DPPTE-AuPEt₃ | 11.65 |
| CholS-AuPEt₃ | 164.4 |
| Et₃PAuCl | 9.736 |
| Auranofin | 5.666 |

OVCAR5 Ovarian Cancer Cells Treated with Au-Salt HDL NPs Decrease Cell Viability

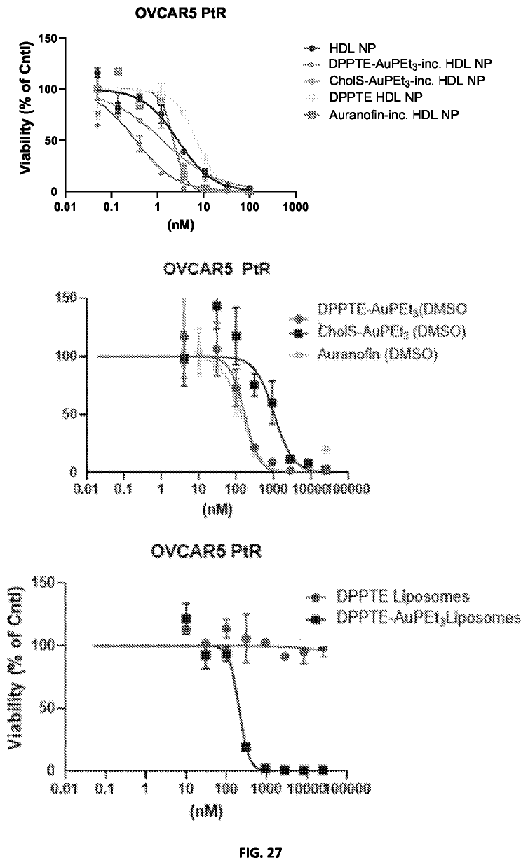

Au-salt containing HDL NPs decrease cell viability more than HDL NPs without Au-salts in OVCAR5 platinum-sensitive (WT) or platinum-resistant (Pt5) cells. OVCAR5 cells (WT or PtR) were treated with gold-bearing HDL-NPs, gold salts, or liposomes as disclosed herein in an assay described in the Materials and protocols for in vitro studies section above. Data are shown in FIG. 26 for WT cells and FIG. 27 for PtR cells (in both figures: gold HDL-NPs—top, gold salts alone—middle, and liposomes containing DPPTE alone, or DPPTE-AuPEt₃—bottom), and IC50 data are presented in Table D (WT) and Table E (PtR):

TABLE D

| NP, Salt, or Liposome tested | IC50 (nM) |
|---|---|
| HDL NP | 133.4 |
| DPPTE HDL NP | 114.6 |
| DPPTE-AuPEt₃-inc. HDL NP | 16.94 |
| Auranofin-inc. HDL NP | 26.95 |
| DPPTE-AuPEt₃ (DMSO) | 3098 |
| Auranofin (DMSO) | 1228 |
| DPPTE Liposomes | N/A |
| DPPTE-AuPEt₃ Liposomes | 4562 |

TABLE E

| NP, Salt, or Liposome tested | IC50 (nM) |
|---|---|
| HDL NP | 2.776 |
| DPPTE HDL NP | 7.130 |
| DPPTE-AuPEt₃-inc. HDL NP | 0.3375 |
| CholS-AuPEt₃-inc. HDL NP | 1.293 |
| Auranofin-inc. HDL NP | 2.225 |
| DPPTE-AuPEt₃ (DMSO) | 170.2 |
| CholS-AuPEt₃ (DMSO) | 1053 |
| Auranofin (DMSO) | 137.8 |
| DPPTE Liposomes | N/A |
| DPPTE-AuPEt₃ Liposomes | 206.3 |

OVCAR5 PtR Ovarian Cancer Cells Treated with Au-Salt HDL NPs have Increased Oxidized Lipids

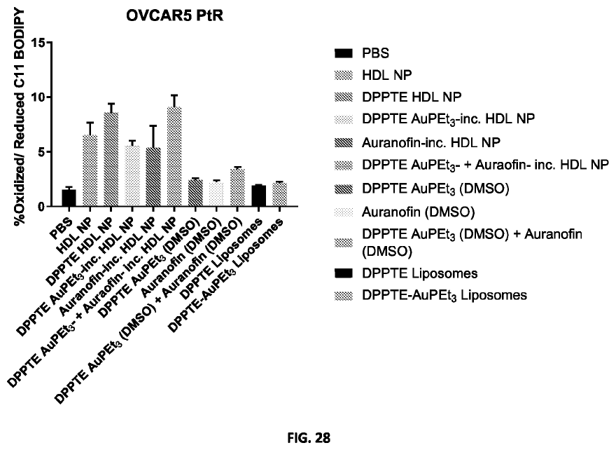

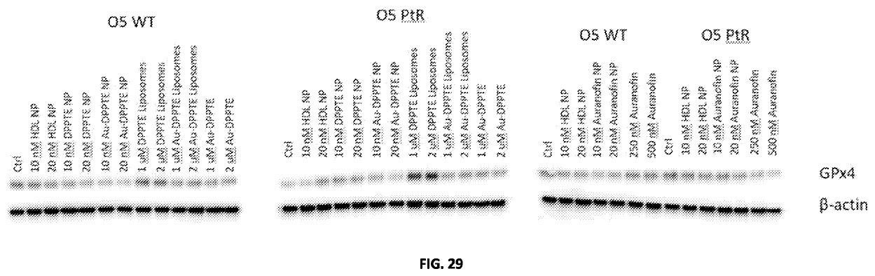

Au-salt containing HDL NPs decrease cell viability in a mechanism consistent with ferroptosis as seen by increased oxidized lipid staining (C11 BODIPY). Au-salts alone do not significantly increase oxidized lipids and, without wishing to be bound by any particular theory, therefore appear to not kill cells by ferroptosis. OVCAR5 PtR cells were treated with gold-bearing HDL-NPs, gold salts, or liposomes as disclosed herein in an assay described in e.g., J. Rink et al., J. Biol. Chem. 296(C):100100 (2021), and Y. Wang et al., Adv. Sci. (Weinh). 11(13):e2305212 (2024). Data are shown in FIG. 28.

OVCAR5 WT and PtR Ovarian Cancer Cells Treated with Au-Salt HDL NPs Decrease GPX4 Protein Expression

Without wishing to be bound by any particular theory, Au-salt containing HDL NPs decrease cell viability in a mechanism consistent with ferroptosis as seen by decreased GPX4 protein expression, and Au-salts alone do not significantly decrease GPX4 protein expression and therefore do not kill cells by ferroptosis. OVCAR5 PtR cells were treated with gold-bearing HDL-NPs, gold salts, or liposomes as disclosed herein in an assay described in e.g., J. Rink et al., J. Biol. Chem. 296(C):100100 (2021) and Y. Wang et al., Adv. Sci. (Weinh). 11(13):e2305212 (2024). Data are shown in FIG. 29.

### Example 9: Au-HDL Nanoparticles Induce Cell Death in Prostate Cancer Models

### DPPTE-AuPEt3 Au-HDL NPs Decrease Cell Viability at 72 hr in AR-Dependent LNCaP, and Castration-Resistant CWR-R1 and 22rv1 Prostate Cancer Cells

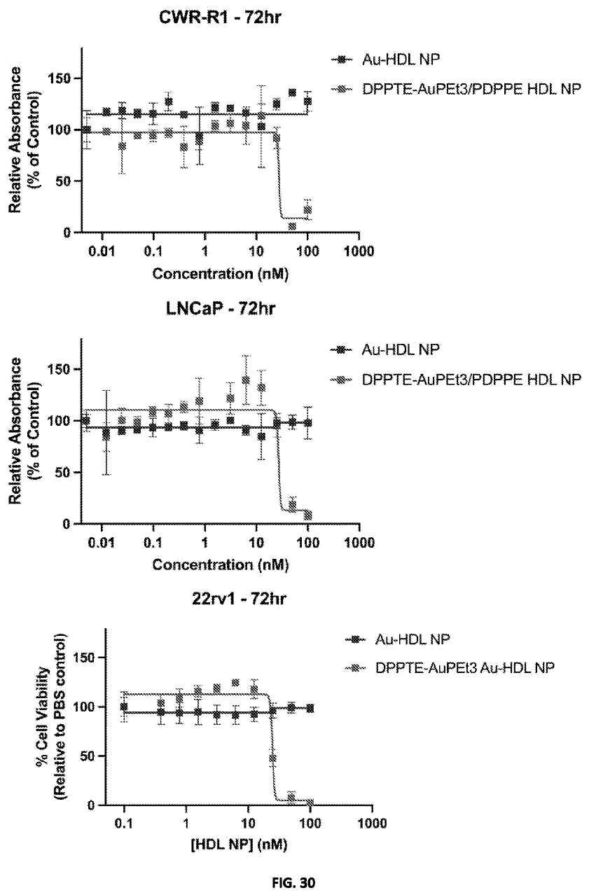

Au-salt containing HDL NPs decrease cell viability in AR-dependent LNCaP, and castration-resistant CWR-R1 and 22rv1 prostate cancer cells, while these prostate cancer cell lines are insensitive to HDL NPs without Au-salts at 72 hr timepoint. Cells were treated with gold-bearing HDL-NPs as disclosed herein in an assay described in the Materials and protocols for in vitro studies section above. Data are shown in FIG. 30.

### DPPTE-AuPEt3 HDL NPs Reduce GPx4 Expression in Castration-Resistant CWR-R1 Prostate Cancer Cells

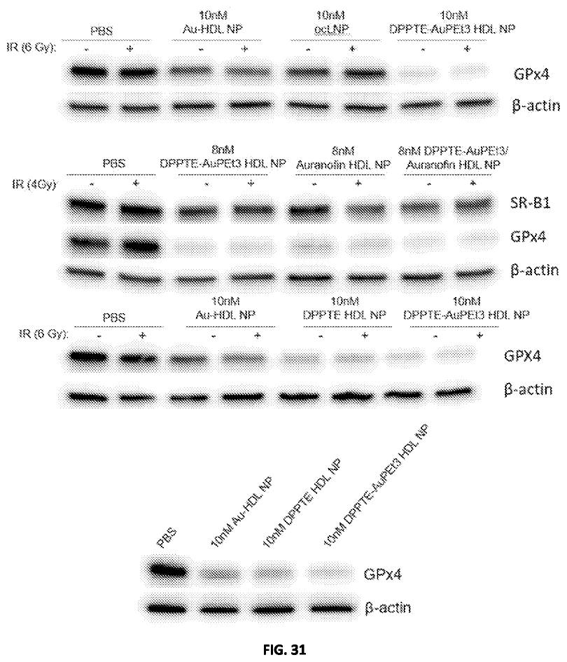

DPPTE-AuPEt3 HDL NPs reduce GPx4 expression in castration-resistant CWR-R1 prostate cancer cells, more so than standard Au-HDL NPs without the addition of DPPTE-AuPEt3 gold salts. Cells were treated with gold-bearing HDL-NPs as disclosed herein and infrared radiation (IR) in an assay described in the Materials and protocols for in vitro studies section above. Data are shown in FIG. 31.

### DPPTE-AuPEt3/PDPPE HDL NPs Sensitize CWR-R1 and 22Rv1 Cells to Ionizing Radiation (IR)

DPPTE-AuPEt3/PDPPE HDL NPs enhance the radiosensitivity of CWR-R1 and 22rv1 cells to ionizing radiation (IR) as shown by decreased clonogenicity/surviving fraction in response radiation as compared to PBS control cells. DPPTE-AuPEt3/PDPPE HDL NPs enhance expression of p21, a marker of cell cycle arrest, in response to IR. Sensitizer enhancement ratio (SER)=2.4 for CWR-R1 cells, and 1.3 for 22rv1 cells. Cells were treated with gold-bearing HDL-NPs as disclosed herein and ionizing radiation (IR).

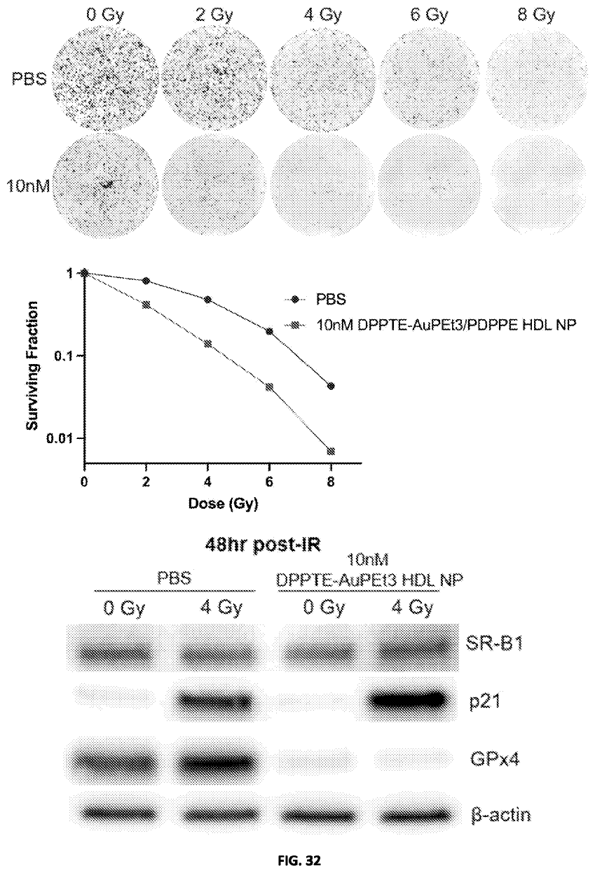

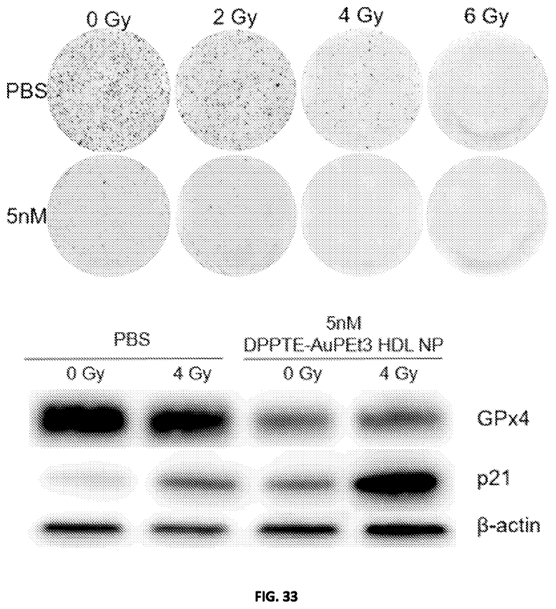

In vitro clonogenic assay. CWR-R1 (700,000 cells/flask) and 22rv1 (700,000 cells/flask) were plated into t25 flasks and incubated for 24 h. Media on the cells was then replaced with pre-prepared media containing the appropriate drug formulations at the appropriate drug concentration for pre-treatment. After 72 h, pre-treated CWR-R1 (4,000 cells/well) and 22rv1 (4,000 cells/well) cells were replated into 6-well plates while continuing drug treatment. The following day, CWR-R1 and 22rv1 cells were irradiated at increasing radiation doses using the RadSource RS-2000 biological research irradiator at Northwestern University. Clonogenic assays were then incubated undisturbed for 9 additional days before fixing cells with 100% methanol (20 min) and staining with 0.01% crystal violet in water (1 h). Colony formation was quantified using ImageJ according to established methods (Harper J. ImageJ plate count protocol. protocolsio. 2016. doi: dx.doi.org/10.17504/protocols.io.f2mbqc6.). Radiation-dose response curves were generated by plotting surviving fractions at increasing radiation doses. Surviving fractions were calculated using the formula: SF=number of colonies formed after IR/(# cells plated×plating efficiency), where plating efficiency PE=# colonies formed/# cells seeded×100%. To calculate sensitizer enhancement ratios (SERs), radiation-dose response curves were fit to a linear-quadratic model using GraphPad PRISM, and the radiation dose for 50% cell survival was extrapolated using the formula S=e\hat{ }(−αD−βD²), where S is the surviving fraction of cells and D is the radiation dose. Protocol adapted from: Franken, N., Rodermond, H., Stap, J. et al. Clonogenic assay of cells in vitro. Nat Protoc 1, 2315-2319 (2006). https://doi.org/10.1038/nprot.2006.339. Data are shown in FIGS. 32 (CWR-R1 cells) and 33 (22rv1 cells).

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A compound, or pharmaceutically acceptable salt thereof, having the structure of Formula I:
R¹—S—Au—P(R²)₃ (I),
wherein
R¹ is a steroidal moiety or a lipid moiety; and
each R² is independently C₁₋₆ alkyl or C₆₋₁₀ aryl.

2. The compound or salt of claim 1, wherein R¹ is a steroidal moiety and the steroidal moiety comprises a sterol derivative.

3. The compound or salt of claim 1, wherein R¹ is a lipid moiety and the lipid moiety comprises a neutral lipid or a phospholipid derivative.

4. The compound or salt of claim 1 or 2, wherein R¹ has the structure:

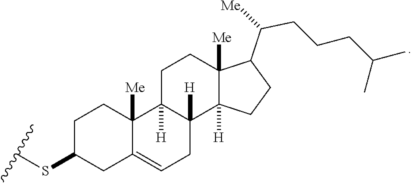

5. The compound or salt of claim 1 or 3, wherein R¹ has the structure: 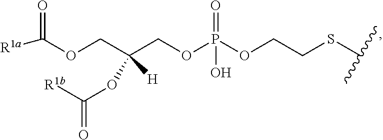
wherein
R^{1a} and R^{1b} are each independently C₄₋₂₂ alkyl or C₄₋₂₂ alkenyl, and wherein the C₄₋₂₂ alkenyl is optionally mono- or poly-unsaturated.

6. The compound or salt of claim 5, wherein R^{1a} and R^{1b} are each independently C₁₂-C₁₈-alkyl.

7. The compound or salt of claim 5 or 6, wherein R¹ has the structure:

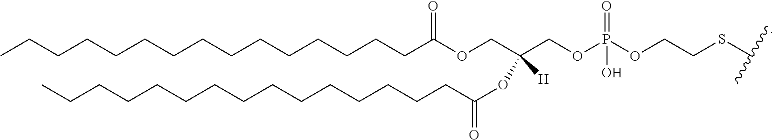 [SMILES: *SCCOP(=O)(O)OC[C@@H](COC(=O)CCCCCCCCCCCCCCC)OC(=O)CCCCCCCCCCCCCCC]

8. The compound or salt of any one of claims 1 to 7, wherein each R² is independently C₁₋₃ alkyl.

9. The compound or salt of any one of claims 1 to 8, wherein at least one R² is ethyl.

10. The compound or salt of any one of claims 1 to 9, wherein each R² is ethyl.

11. A compound, or pharmaceutically acceptable salt thereof, having a structure as shown in Table A.

12. A microparticle or nanoparticle comprising the compound or salt of any one of claims 1 to 11 and a lipid nanoparticle (LN) or a synthetic lipoprotein nanoparticle (SLN).

13. The microparticle or nanoparticle of claim 12, wherein the synthetic lipoprotein nanoparticle (SLN) is a high-density lipoprotein-like nanoparticle (HDL-NP) comprising:
(a) an organic or an inorganic nanoparticle core;
(b) a shell surrounding and/or attached to the core wherein the shell comprises one or more apolipoproteins and one or more phospholipids; and
(c) wherein the compound or salt of any one of claims 1 to 11 is associated with one or more of the core or shell.

14. The microparticle or nanoparticle of claim 13, wherein the core is an organic core.

15. The microparticle or nanoparticle of claim 14, wherein the organic core comprises a dendritic scaffold.

16. The microparticle or nanoparticle of claim 15, wherein the dendritic scaffold has one or more lipid-conjugated arms.

17. The microparticle or nanoparticle of claim 15 or 16, wherein the dendritic scaffold is a hydrophobic phospholipid-conjugated scaffold.

18. The microparticle or nanoparticle of claim 17, wherein the dendritic scaffold comprises one or more headgroup-modified phospholipids.

19. The microparticle or nanoparticle of claim 17 or 18, comprising four headgroup-modified phospholipids (PL₄).

20. The microparticle or nanoparticle of claim 19, wherein the dendritic scaffold has the structure:

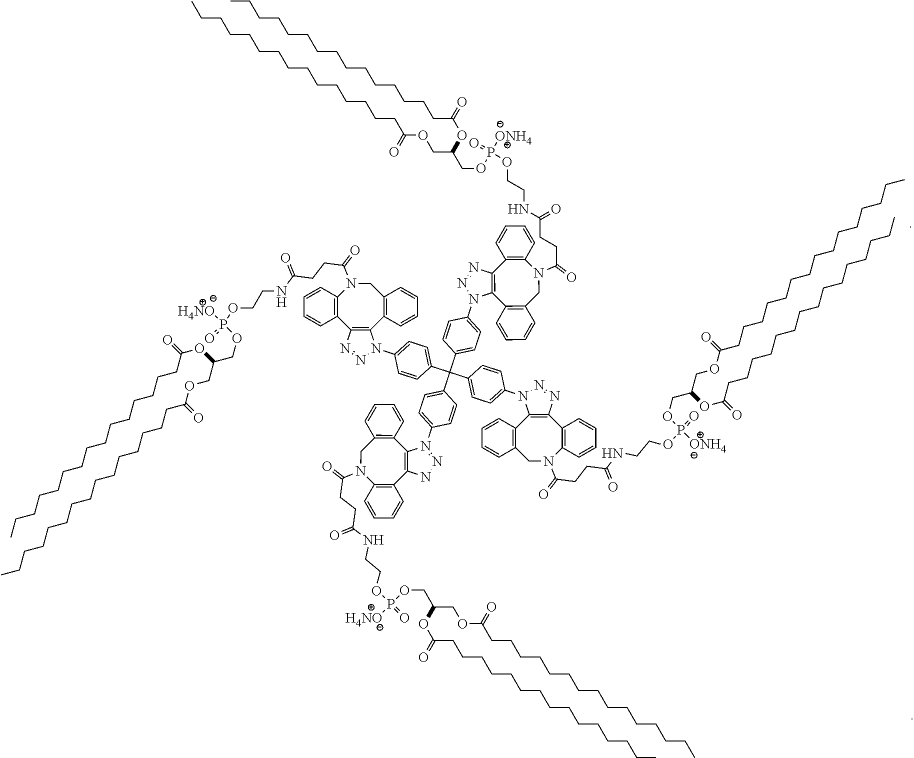

21. The microparticle or nanoparticle of claim 15 or 16, wherein the dendritic scaffold comprises four 3-(stearoyloxy)-2,2-bis[(stearoyloxy)methyl]propyl stearate (PE-S₄).

22. The microparticle or nanoparticle of claim 21, wherein the dendritic scaffold has the structure:

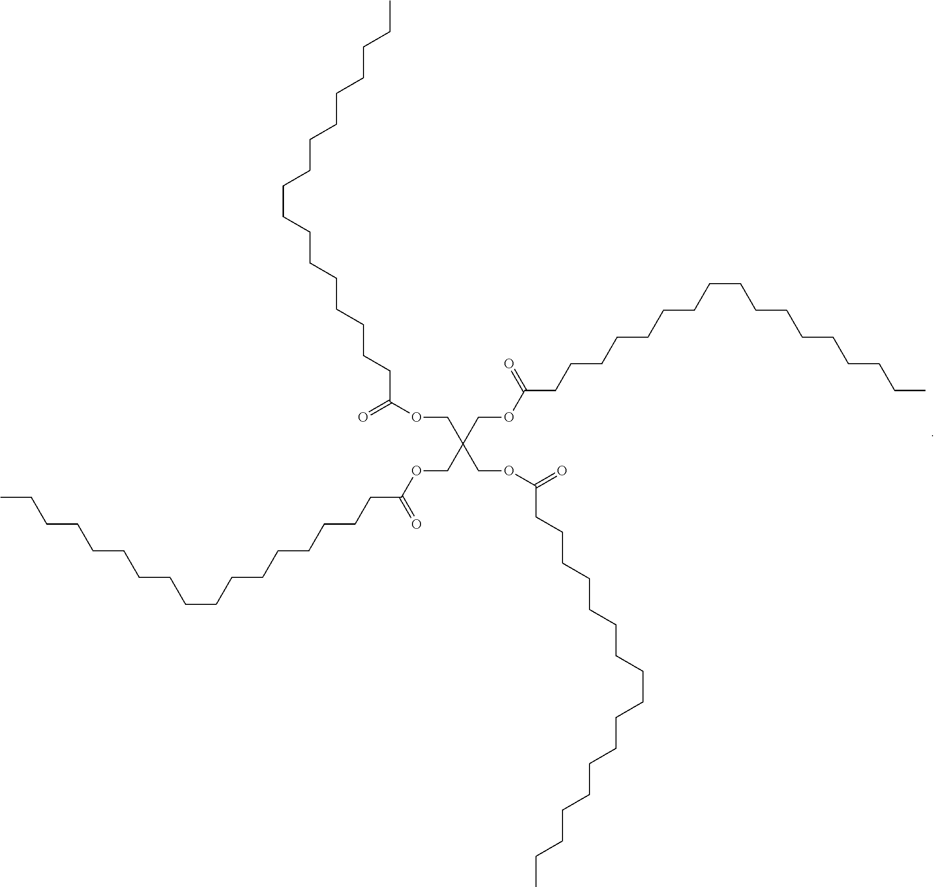 [SMILES: CCCCCCCCCCCCCCCCCC(=O)OCC(COC(=O)CCCCCCCCCCCCCCCCC)(COC(=O)CCCCCCCCCCCCCCCCC)COC(=O)CCCCCCCCCCCCCCCCC]

23. The microparticle or nanoparticle of claim 13, wherein the core is an inorganic nanoparticle core.

24. The microparticle or nanoparticle of claim 23, wherein the core is a gold nanoparticle core.

25. The microparticle or nanoparticle of any one of claims 13 to 24, wherein the one or more apolipoproteins comprise apolipoprotein A-I (apoA-I) or apolipoprotein E (apo E).

26. The microparticle or nanoparticle of any one of claims 14 to 22, wherein the compound or salt of any one of claims 1 to 11 is associated to the organic core, shell, and/or apolipoprotein non-covalently or through hydrophobic interactions.

27. The microparticle or nanoparticle of any one of claims 13 to 26, wherein the shell is attached to the core non-covalently.

28. The microparticle or nanoparticle of any one of claims 13 to 27, wherein the shell is attached to the core through hydrophobic interactions.

29. The microparticle or nanoparticle of any one of claims 13 to 28, wherein the shell is a lipid shell, wherein the lipid shell is a lipid monolayer or a lipid bilayer.

30. The microparticle or nanoparticle of any one of claims 13 to 29, wherein the one or more phospholipids comprise 1,2-dipalmitoyl-sn-glycero-3-phosphoethanolamine-N-[3-(2-pyridyldithio)propionate] (PDP-PE), 1,2-dipalmitoyl-sn-glycero-3-phosphocholine (DPPC), or a combination thereof.

31. The microparticle or nanoparticle of any one of claims 13 to 30, further comprising one or more neutral lipids.

32. The microparticle or nanoparticle of any one of claims 13 to 31, wherein the HDL-NP has a diameter of about 5 nm to about 150 nm.

33. The microparticle or nanoparticle of claim 32, wherein the HDL-NP has a diameter of about 5 nm to about 30 nm, about 5 nm to about 20 nm, about 5 nm to about 15 nm, about 5 nm to about 10 nm, about 8 nm to about 13 nm, about 8 nm to about 12 nm, or about 10 nm.

34. The microparticle or nanoparticle of any one of claims 13 to 33, wherein the HDL-NP has a zeta potential of −16 to −60 mV.

35. The microparticle or nanoparticle of any one of claims 13 to 34, wherein the HDL-NP further comprises one or more therapeutic agents.

36. The microparticle or nanoparticle of claim 35, wherein the one or more therapeutic agents is an anti-inflammatory, anti-oxidants, anti-microbial, anti-viral, anti-cancer agents, or polyunsaturated fatty acids, or a combination thereof.

37. The microparticle or nanoparticle of claim 35 or 36, wherein the one or more therapeutic agents comprise auranofin, PIK75 (F7) (C₁₆H₁₄BrN₅O₄S·HCl), doxorubicin, vincristine, gemcitabine, paclitaxel, docetaxel, andrographolide, sutent, tamoxifen, vitamin D, vitamin E, or a combination thereof.

38. A pharmaceutical composition comprising the compound or salt of any one of claims 1 to 11 or the microparticle or nanoparticle of any one of claims 12 to 37 and a pharmaceutically acceptable carrier or excipient.

39. A method of treating or preventing a disease or disorder comprising administering to a subject in need thereof a therapeutically effective amount of the compound or salt of any one of claims 1 to 11, the microparticle or nanoparticle of any one of claims 12 to 37, or the pharmaceutical composition of claim 38.

40. The method of claim 39, wherein the disease or disorder is an inflammatory disease, infectious disease, or cancer.

41. The method of claim 40, wherein the inflammatory disease is psoriasis, psoriatic arthritis, atopic dermatitis, Sjogren's syndrome, multiple sclerosis, amyotrophic lateral sclerosis, neuroinflammation, carditis, myocarditis, angiitis, arteritis, nephritis, neuritis, pleuritis, pneumonitis, arthritis, polyarthritis, optic neuritis, ophthalmitis, meningitis, myalgia, polymyalgia, polymyalgia rheumatica, gastroenteritis, enteritis, esophagitis, Crohn's disease, ulcerative colitis, colitis, proctitis, interstitial cystitis, cystitis, irritable bowel syndrome, dermatitis, mucositis, urethritis, or prostatitis.

42. The method of claim 41, wherein the arthritis is rheumatoid arthritis.

43. The method of claim 40, wherein the cancer is sarcoma, uterine cancer, cervical cancer, lung cancer, renal cancer, leukemia, lymphoma, multiple myeloma, melanoma, ovarian cancer, breast cancer, gastrointestinal malignancies, brain tumors, prostate cancer, or colon cancer.

44. A method for reducing inflammation comprising administering to a subject in need thereof a therapeutically effective amount of the compound or salt of any one of claims 1 to 11, the microparticle or nanoparticle of any one of claims 12 to 37, or the pharmaceutical composition of claim 38.

45. The method of claim 44, wherein the inflammation is caused by a noninfectious disease.

46. The method of claim 40, wherein the infectious disease is a parasitic disease, a bacterial disease, a fungal disease, or a viral disease.

47. A method for inhibiting neoplastic cell growth comprising administering to a subject in need thereof a therapeutically effective amount of the compound or salt of any one of claims 1 to 11, the microparticle or nanoparticle of any one of claims 12 to 37, or the pharmaceutical composition of claim 38.

48. The method of any one of claims 39 to 47, wherein the administering comprises intrathecal, intravenous, intraperitoneal, oral, topical, ocular, intratumoral, or intra-articular administration.

49. The method of any one of claims 39 to 48, further comprising subjecting the subject to radiation in order to treat or prevent the disease or disorder, reduce inflammation, or inhibit neoplastic cell growth.

50. The method of claim 49, wherein the radiation is administered in an amount of at least 2 Gy, at least 4 Gy, at least 6 Gy, at least 8 Gy, or at least 10 Gy.

51. The method of claim 50, wherein the radiation is administered in an amount of at least 2 Gy.

52. The method of any one of claims 49 to 51, wherein the radiation is ionizing radiation.

53. A process of synthesizing a compound of Formula (I):
R¹—S—Au—P(R²)₃ (I), comprising
admixing a compound of Formula (I′):
R¹—SH (I′) and a compound of Formula (I″):
X—Au—P(R²)₃ (I″) in the presence of a base to form the compound of Formula (I),
wherein
R¹ is a steroidal moiety or a lipid moiety;
each R² is independently C₁₋₆ alkyl or C₆₋₁₀ aryl; and
X is a halogen ion.

54. The process of claim 53, wherein the steroidal moiety comprises a sterol derivative.

55. The process of claim 53, wherein the lipid moiety comprises a neutral lipid or a phospholipid derivative.

56. The process of claim 53 or 54, wherein R¹ has the structure:

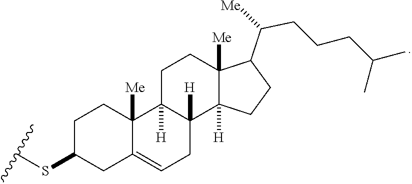

57. The process of claim 53 or 55, wherein R¹ has the structure: 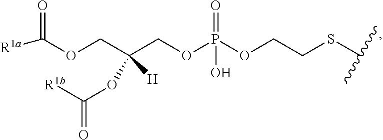
wherein
R^{1a} and R^{1b} are each independently C₄₋₂₂ alkyl or C₄₋₂₂ alkenyl, and wherein the C₄₋₂₂ alkenyl is optionally mono- or poly-unsaturated.

58. The process of claim 57, wherein R^{1a} and R^{1b} are each independently C₁₂-C₁₈-alkyl.

59. The process of claim 57 or 58, wherein R¹ has the structure:

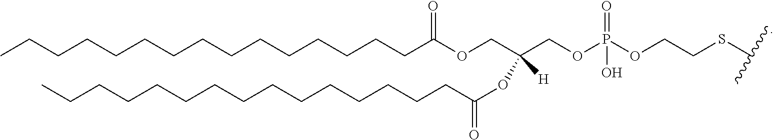 [SMILES: *SCCOP(=O)(O)OC[C@@H](COC(=O)CCCCCCCCCCCCCCC)OC(=O)CCCCCCCCCCCCCCC]

60. The process of any one of claims 53 to 59, wherein each R² is independently C₁₋₃ alkyl.

61. The process of any one of claims 53 to 60, wherein at least one R² is ethyl.

62. The process of any one of claims 53 to 61, wherein each R² is ethyl.

63. The process of any one of claims 53 to 62, wherein the base is an alkali carbonate.

64. The process of claim 63, wherein the base is potassium carbonate.

65. The process of any one of claims 53 to 64, wherein X is chloride.

66. A process of synthesizing the microparticle or nanoparticle of any one of claims 13 to 36, comprising:
(i) admixing one or more gold nanoparticles (AuNPs) and one or more apolipoproteins to form one or more Apo-AuNPs;
(ii) incubating the one or more Apo-AuNPs;
(iii) admixing the one or more Apo-AuNPs and a compound of Formula (I′):
R¹—SH (I′) to form one or more R¹S-Apo-AuNPs;
(iv) incubating the one or more R¹S-Apo-AuNPs; and
(v) admixing the one or more R¹S-Apo-AuNPs and a compound of Formula (I″):
X—Au—P(R²)₃ (I″) in the presence of a base to form the microparticle or nanoparticle,
wherein
X is a halogen ion.

67. The process of claim 66, wherein the one or more apolipoproteins is apolipoprotein A-I (apoA-I) or apolipoprotein E (apo E).

68. The process of claim 66 or 67, wherein X is chloride.

69. The process of claim any one of claims 66 to 68, wherein the base is an alkali carbonate.

70. The process of claim 69, wherein the base is potassium carbonate.

71. The process of any one of claims 66 to 70, wherein incubating the one or more Apo-AuNPs occurs at a temperature of about 5 °C to about 35 °C.

72. The process of claim 71, wherein incubating the one or more Apo-AuNPs occurs at a temperature of about 20 °C to about 25 °C.

73. The process of any one of claims 66 to 72, wherein incubating the one or more Apo-AuNPs occurs for about 8 to about 18 hours.

74. The process of any one of claims 66 to 73, wherein incubating the one or more R¹S-Apo-AuNPs occurs at a temperature of about 5 °C to about 35 °C.

75. The process of claim 74, wherein incubating the one or more R¹S-Apo-AuNPs occurs at a temperature of about 20 °C to about 25 °C.

76. The process of any one of claims 66 to 75, wherein incubating the one or more R¹S-Apo-AuNPs occurs for about 8 to about 18 hours.

77. The process of any one of claims 66 to 76, further comprising the step of incubating the microparticle or nanoparticle.

78. The process of claim 77, wherein incubating the microparticle or nanoparticle occurs at a temperature of about 5 °C to about 35 °C.

79. The process of claim 78, wherein incubating the microparticle or nanoparticle occurs at a temperature of about 20 °C to about 25 °C.

80. The process of claim 78 or 79, wherein incubating the microparticle or nanoparticle occurs for about 8 to about 18 hours.

81. A process of synthesizing the microparticle or nanoparticle of any one of claims 13 to 36, comprising:
(i) admixing one or more gold nanoparticles (AuNPs) and one or more apolipoproteins in a solvent to form one or more Apo-AuNPs;
(ii) incubating the one or more Apo-AuNPs;
(iii) admixing the one or more Apo-AuNPs and one or more phospholipids in an organic solvent to form one or more PL-Apo-AuNPs;
(iv) admixing the one or more PL-Apo-AuNPs and a compound of Formula (I) in the organic solvent; and
(v) removing the organic solvent to form the microparticle or nanoparticle.

82. The process of claim 81, wherein the one or more apolipoproteins is apolipoprotein A-I (apoA-I) or apolipoprotein E (apo-E).

83. The process of claim 81 or 82, wherein the one or more phospholipids is dissolved in an organic solvent prior to admixing in step (iii).

84. The process of claim 83, wherein the organic solvent is a chlorinated solvent.

85. The process of claim 84, wherein the organic solvent is CH₂Cl₂.

86. The process of any one of claims 81 to 85, wherein the one or more phospholipids comprise 1,2-dipalmitoyl-sn-glycero-3-phosphoethanolamine-N-[3-(2-pyridyldithio)propionate] (PDP-PE).

87. The process of any one of claims 81 to 86, wherein incubating the one or more Apo-AuNPs occurs at a temperature of about 5 °C to about 35 °C.

88. The process of claim 87, wherein incubating the one or more Apo-AuNPs occurs at a temperature of about 20 °C to about 25 °C.

89. The process of any one of claims 81 to 87, wherein incubating the one or more Apo-AuNPs occurs for about 1 hour.

90. A process of synthesizing the microparticle or nanoparticle of any one of claims 13 to 36, comprising:
(i) admixing one or more gold nanoparticles (AuNPs) and one or more apolipoproteins in a solvent to form one or more Apo-AuNPs;
(ii) incubating the one or more Apo-AuNPs;
(iii) admixing the one or more Apo-AuNPs and one or more phospholipids to form one or more PL-Apo-AuNPs;
(iv) incubating the one or more one or more PL-Apo-AuNPs;
(v) optionally repeating steps (ii) and (iii) to incorporate one or more additional phospholipids into the PL-Apo-AuNPs; and
(iv) admixing the one or more PL-Apo-AuNPs and aqueous KCN to form the microparticle or nanoparticle.

91. The process of claim 90, wherein the one or more apolipoproteins is apolipoprotein A-I (apoA-I) or apolipoprotein E (apo E).

92. The process of claim 90 or 91, wherein the one or more phospholipids is dissolved in an organic solvent prior to admixing in step (iii).

93. The process of claim 92, wherein the organic solvent is a polar organic solvent.

94. The process of claim 93, wherein the organic solvent is ethanol.

95. The process of any one of claims 90 to 94, wherein the one or more phospholipids comprise 1,2-dipalmitoyl-sn-glycero-3-phosphoethanolamine-N-[3-(2-pyridyldithio)propionate] (PDP-PE).

96. The process of any one of claims 90 to 95, wherein incubating the one or more Apo-AuNPs occurs at a temperature of about 5 °C to about 35 °C.

97. The process of claim 96, wherein incubating the one or more Apo-AuNPs occurs at a temperature of about 20 °C to about 25 °C.

98. The process of any one of claims 90 to 97, wherein incubating the one or more Apo-AuNPs occurs for about 1 hour.

99. The process of any one of claims 90 to 98, wherein incubating the one or more PL-Apo-AuNPs occurs at a temperature of about 5 °C to about 35 °C.

100. The process of claim 99, wherein incubating the one or more PL-Apo-AuNPs occurs at a temperature of about 20 °C to about 25 °C.

101. The process of any one of claims 90 to 100, wherein incubating the one or more PL-Apo-AuNPs occurs for about 1 hour.

102. The process of any one of claims 90 to 101, further comprising the step of incubating the microparticle or nanoparticle.

103. The process of claim 102, wherein incubating the microparticle or nanoparticle occurs at a temperature of about 5 °C to about 35 °C.

104. The process of claim 103, wherein incubating the microparticle or nanoparticle occurs at a temperature of about 20 °C to about 25 °C.

105. The process of claim 103 or 104, wherein incubating the microparticle or nanoparticle occurs for about 8 to about 18 hours.

106. A process of synthesizing the microparticle or nanoparticle of any one of claims 13 to 36, comprising admixing a dendritic scaffold, an apolipoprotein, a sterol salt, a phospholipid, and a compound of Formula (I) to form the microparticle or nanoparticle.

107. The process of claim 106, wherein the dendritic scaffold is a pentaerythrityl tetraester.

108. The process of claim 107, wherein the dendritic scaffold is 3-(stearoyloxy)-2,2-bis[(stearoyloxy)methyl]propyl stearate (PE-S₄).

109. The process of any one of claims 106 to 108, wherein the apolipoprotein is apolipoprotein A-I (apoA-I) or apolipoprotein E (apo E).

110. The process of any one of claims 106 to 109, wherein the sterol salt is a cholate salt.

111. The process of claim 110, wherein the cholate salt is sodium cholate.

112. The process of any one of claims 106 to 111, wherein the phospholipid is 1,2-dioleoyl-sn-glycero-3-phosphocholine (DOPC).

113. The process of any one of claims 106 to 112, wherein the ratio of the phospholipid and compound of Formula (I) to the dendritic scaffold is about 300:1.

114. The process of any one of claims 106 to 113, further comprising the step of incubating the microparticle or nanoparticle.

115. The process of claim 114, wherein incubating the microparticle or nanoparticle occurs at a temperature of about 5 °C to about 35 °C.

116. The process of claim 115, wherein incubating the microparticle or nanoparticle occurs at a temperature of about 20 °C to about 25 °C.

117. The process of any one of claims 114 to 116, wherein incubating the microparticle or nanoparticle occurs for about 8 to about 18 hours.

118. A process of synthesizing the microparticle or nanoparticle of any one of claims 13 to 36, the method comprising administering at least the following components to a mammalian subject:
an organic or an inorganic nanoparticle core; and
at least one compound having the structure of Formula I;
wherein assembly of the components into the microparticle or nanoparticle occurs in vivo in the mammalian subject, said microparticle or nanoparticle, when assembled, including apolipoprotein from the mammalian subject.

119. A process of synthesizing the microparticle or nanoparticle of any one of claims 13 to 36, the method comprising administering at least the following components to a mammalian subject:
an organic or an inorganic nanoparticle core;
at least one compound having the structure of Formula I; and
at least one apolipoprotein;
wherein assembly of the components into the microparticle or nanoparticle occurs in vivo in the mammalian subject.

120. The microparticle or nanoparticle of any one of claims 12 to 37, or the pharmaceutical composition of claim 38, for use in the treatment or prevention of inflammation.

121. The microparticle or nanoparticle of any one of claims 12 to 37, or the pharmaceutical composition of claim 38, for use in the treatment of cancer, a neoplastic disorder, or an infection.

122. A method for reducing, inducing, or increasing ferroptosis in a cell, comprising contacting the cell with an effective amount of the compound or salt of any one of claims 1 to 11, the microparticle or nanoparticle of any one of claims 12 to 37, or the pharmaceutical composition of claim 38.

123. The method of claim 122, wherein the contacting is performed in vivo.

124. The method of claim 122 or 123, wherein reducing, inducing, or increasing ferroptosis in a cell treats or prevents a disease or disorder in a subject.

125. The method of claim 124, wherein the disease or disorder is cancer.

126. The method of claim 125, wherein the cancer is ovarian cancer.

127. The method of claim 122, wherein the cell is a cancerous cell.
